(19) United States
(12) Patent Application Publication
(10) Pub. No.: US 2022/0265256 A1
(43) Pub. Date: Aug. 25, 2022

(54) MEDICAL RETRACTOR ASSEMBLY AND GRAFT INSERTER APPARATUS

(71) Applicant: Scuderia Minimo LLC, Jenks, OK (US)

(72) Inventors: Fernando L. Villamil, Jenks, OK (US); Leighton J. LaPierre, Hampstead, NC (US); Brandon Arthurs, Wilmington, NC (US)

(21) Appl. No.: 17/668,350

(22) Filed: Feb. 9, 2022

Related U.S. Application Data
(60) Provisional application No. 63/147,344, filed on Feb. 9, 2021.

(51) Int. Cl. A61B 17/02; A61F 2/46

(52) U.S. Cl. CPC A61B 17/0206; A61F 2/4601; A61F 2/4611; A61B 2017/0256; A61B 2017/00367

(57) ABSTRACT

A medical retractor having a first retractor blade with a shim channel disposed in the first retractor blade. The shim channel has a cross-sectional area which remains unchanged for an entire length of the shim channel. The unchanged cross-sectional area of the shim channel permits medical tools to slide therethrough to engage a surgical site. Or, this permits the medical retractor to slide down over the medical tools that are engaged with the surgical site. Also, a medical retractor assembly that includes a medical retractor having a first retractor blade with a shim channel disposed in the first retractor blade. The medical retractor assembly also includes a first dilator having a cylindrical component and a shim component. The shim component can slidably engage with the shim channel of the medical retractor. The first dilator has a cross-section for most of its length that has a round portion and an arc-shaped portion attached to the round portion.

### STATEMENT REGARDING FEDERALLY SPONSORED RESEARCH OR DEVELOPMENT

Not applicable.

### CROSS-REFERENCE TO RELATED APPLICATIONS

The present application is a conversion of U.S. Provisional Application having U.S. Ser. No. 63/147,344, filed Feb. 9, 2021, which claims the benefit under 35 U.S.C. 119(e). The disclosure of which is hereby expressly incorporated herein by reference.

### BACKGROUND OF THE DISCLOSURE

### 1. Field of the Invention

The present disclosure relates to a medical retractor assembly that is multi-use for various surgical approaches to the spine and enables safer and more reproducible results. The disclosure is also related to a biological graft inserter apparatus for more efficiently placing a biological graft and implant between discs. The present disclosure is also directed towards methods of using the medical retractor assembly and the graft inserter apparatus.

### 2. Description of the Related Art

Current medical retractors have various limitations, such as limited exposure area for placing various implant sizes, or do not provide direct access to discs. For example, with certain medical retractors, the surgeon must insert a portion of the medical retractor and then rotate the medical retractor. In other instances, the surgeon must insert the retractor over a series of dilators and then remove the dilators to deliver a temporary stabilizing fixation device to the spine. Some medical retractors are better for lateral lumbar interbody fusions (LLIFs), and some are better for transforaminal lumbar interbody fusions (TLIFs). Additionally, some spinal retractors are better for LLIFs or TLIFs with the patient in various positions such as lateral decubitus or prone. A number of the traditional medical retractors pose an increased risk for nerve damage or imprecise targeting to determine precise location in relation to the spinal anatomy.

Accordingly, there is a need for a safer medical retractor that can be used for both LLIFs and TLIFs and provide safer and more reproducible results. There is also a need for a biological graft inserter apparatus that can be used with the medical retractor assembly.

### SUMMARY OF THE DISCLOSURE

The present disclosure is directed to a medical retractor having a first retractor blade with a shim channel disposed in the first retractor blade. The shim channel has a cross-sectional area which remains unchanged for an entire length of the shim channel. The unchanged cross-sectional area of the shim channel permits medical tools to slide therethrough to engage a surgical site. Or, this permits the medical retractor to slide down over themedical tools that are engaged with the surgical site.

The present disclosure is also directed to a medical retractor assembly that includes a medical retractor having a first retractor blade with a shim channel disposed in the first retractor blade. The medical retractor assembly also includes a first dilator having a cylindrical component and a shim component. The shim component can slidably engage with the shim channel of the medical retractor. The first dilator has a cross-section for most of its length that has a round portion and an arc-shaped portion attached to the round portion.

### BRIEF DESCRIPTION OF THE DRAWINGS

FIG. 1 is a perspective view of two medical retractor assemblies constructed in accordance with the present disclosure.

FIG. 2A is a perspective view of a medical retractor in a closed position constructed in accordance with the present disclosure.

FIG. 2B is a perspective view of the medical retractor in an open position constructed in accordance with the present disclosure.

FIGS. 3A-3E are various views of the medical retractor constructed in accordance with the present disclosure.

FIGS. 3F-3G are exploded views of various portions of the medical retractor constructed in accordance with the present disclosure.

FIG. 4A is an exploded view of a portion of the medical retractor constructed in accordance with the present disclosure.

FIG. 4B is a side elevation view of a portion of the medical retractor constructed in accordance with the present disclosure.

FIGS. 5A-5C are various views of one embodiment of a retractor blade constructed in accordance with the present disclosure.

FIGS. 5D-5E are various views of another embodiment of a retractor blade constructed in accordance with the present disclosure.

FIGS. 6A-6E are various views of a dilator of the retractor blade assembly constructed in accordance with the present disclosure.

FIGS. 7A-7B are various views of another dilator of the retractor blade assembly constructed in accordance with the present disclosure.

FIGS. 8A-8B are various views of yet another dilator of the retractor blade assembly constructed in accordance with the present disclosure.

FIGS. 8C-8D are various views of the dilators and/or the dilators and retractor blades of the retractor blade assembly constructed in accordance with the present disclosure.

FIGS. 9A-9B are various views of a threaded fixation apparatus of the retractor blade assembly constructed in accordance with the present disclosure.

FIGS. 10A-10D are various views of a shim fixation apparatus of the retractor blade assembly constructed in accordance with the present disclosure.

FIGS. 11A-11D are various views of a shim tool of the retractor blade assembly constructed in accordance with the present disclosure.

FIGS. 12A-12C are various views of an inserter apparatus constructed in accordance with the present disclosure.

FIGS. 13A-13C are various views of the inserter apparatus shown in use and constructed in accordance with the present disclosure.

FIG. 14 is a perspective view of another embodiment of an inserter apparatus constructed in accordance with the present disclosure.

### DETAILED DESCRIPTION OF THE DISCLOSURE

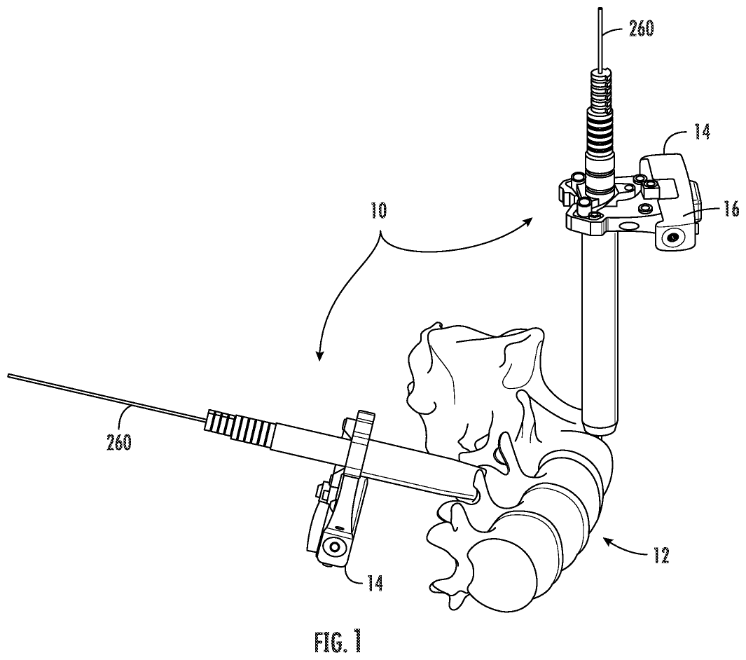

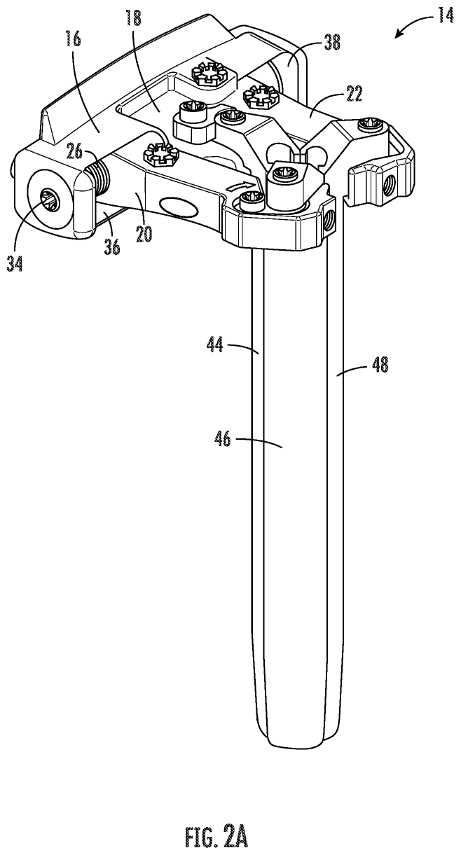

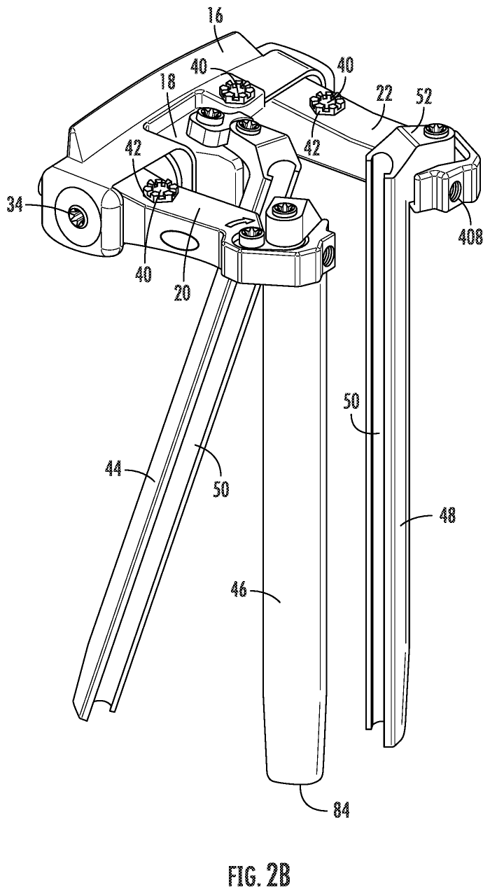

The present disclosure relates to a medical retractor assembly 10 for use by doctors to perform various surgeries near the spine 12. Referring now to FIGS. 1-2B, the medical retractor assembly 10 can include a medical retractor 14. The medical retractor 14 includes a retractor body 16, a middle arm 18, a first outer arm 20 and a second outer arm 22. The middle arm 18 can move toward and away from the retractor body 16 along a first axis and within a portion of the retractor body 16. The first and second outer arms 20 and 22 each extend partially inside the retractor body 16 and they extend away from the retractor body 16, out beyond the middle arm 18. The first and second outer arms 20 and 22 can move laterally toward and away from each other and toward and away from the middle arm 18 along a second axis that is perpendicular to the first axis. The first and second arms 20 and 22 can move independently of each other (i.e., one arm at a time can move toward or away from the other arm) or they can move concurrently (i.e., both the first and second outer arms 20 and 22 moving at the same time, either toward each other or away).

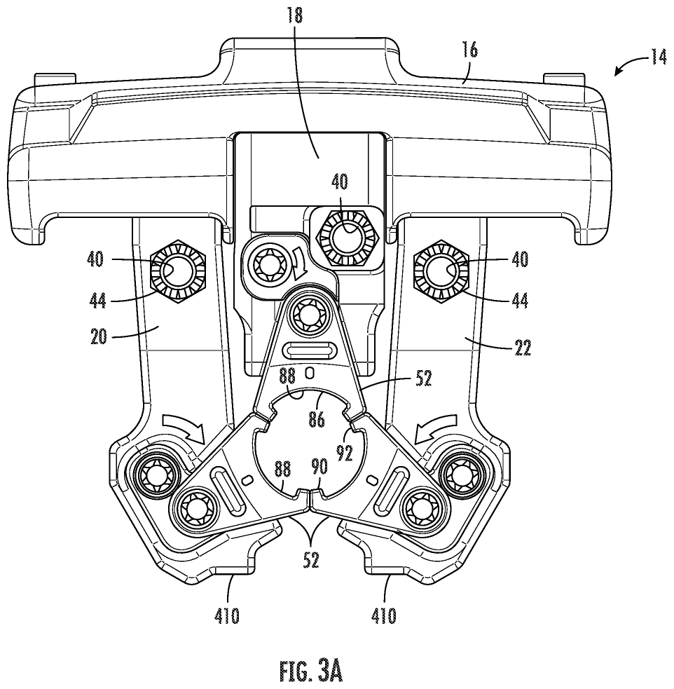

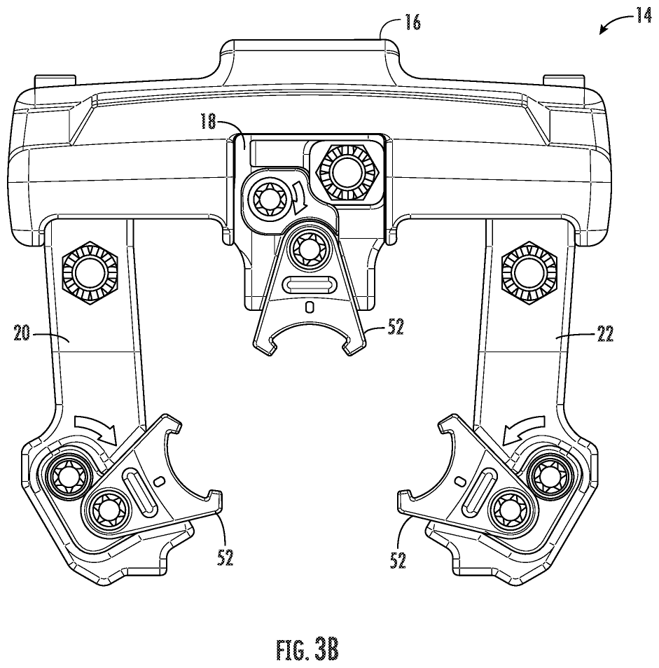

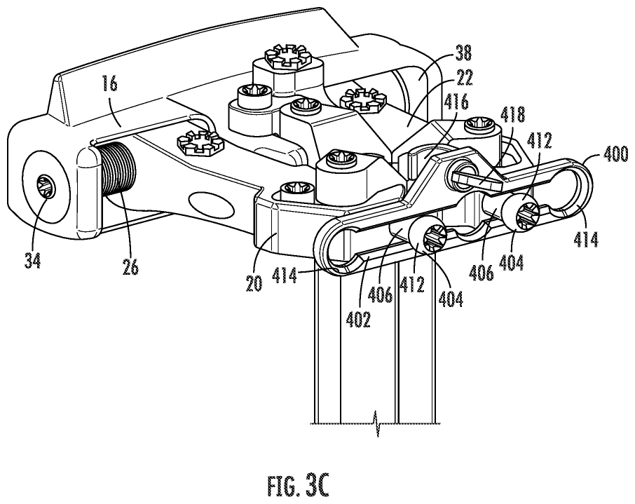

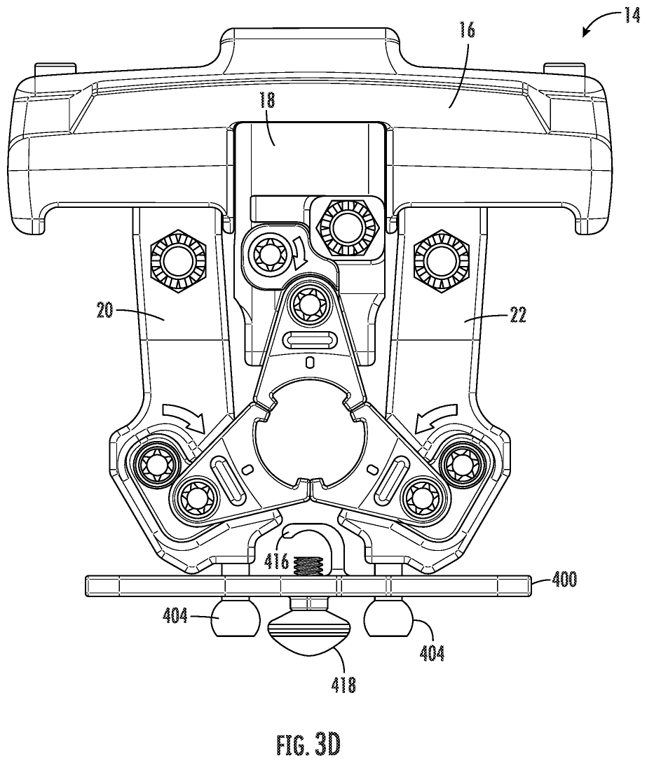

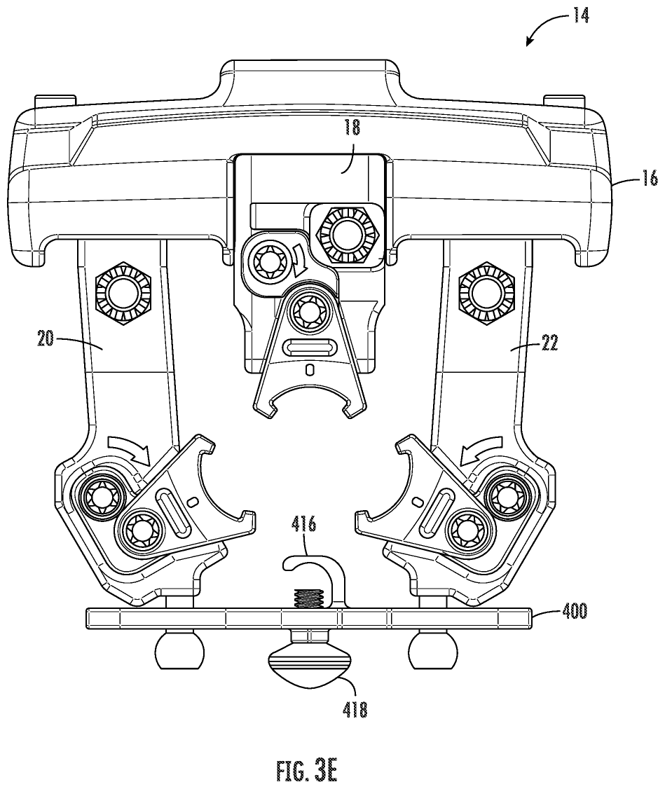

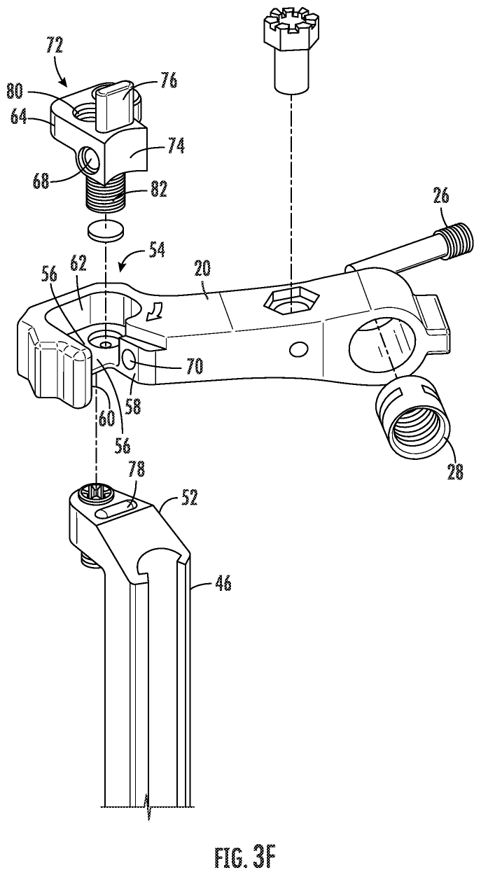

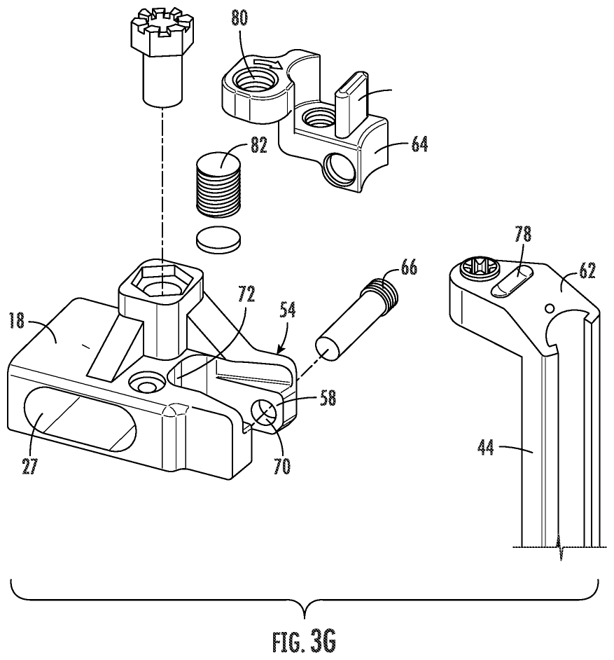

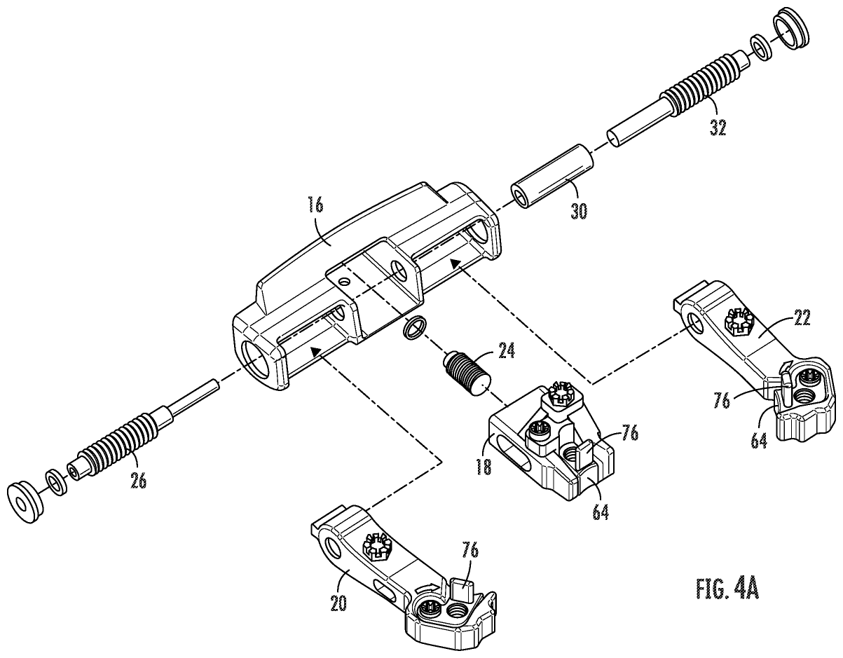

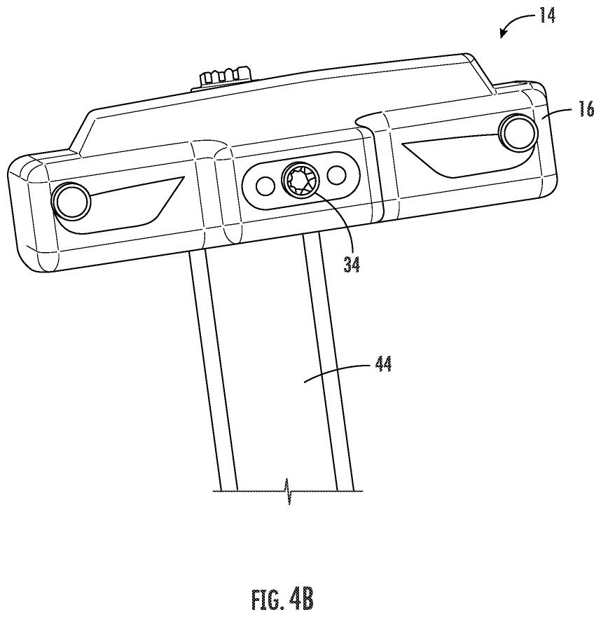

Referring now to FIGS. 3A-4B, the arms 18, 20 and 22 of the medical retractor 14 can be moved by any means known in the art. In one embodiment, the retractor body 16 of the medical retractor 14 can include a first threaded shaft 24 that engages the middle arm 18. When the first threaded shaft 24 is turned, depending on turn direction, the middle arm 18 moves toward or away from the retractor body 16. In this embodiment, the medical retractor 14 can also include a second threaded shaft 26 that engages the first and second outer arms 20 and 22 to move them. In this embodiment, the second threaded shaft 26 passes through a slot 27 in the middle arm 18 that allows the middle arm 18 to move toward or away from the retractor body 16 and second threaded shaft 26. The first outer arm 20 can have a threaded opening 28 therein to receive the second threaded shaft 26. Similarly, the second outer arm 22 can have a threaded opening 30 disposed therein for receiving the second threaded shaft 26. The second threaded shaft 26 can be threaded such that when the second threaded shaft 26 is turned one direction, the first and second outer arms 20 and 22 move away from each other, and when turned the other direction, the first and second outer arms 20 and 22 move toward each other. In another embodiment, the medical retractor 14 has a second threaded shaft 26 that engages only the first outer arm 20, which can move it independently of the middle arm 18 and the second outer arm 22. In this embodiment, the medical retractor 14 can include a third threaded shaft 32 that engages only the second outer arm 22, which can move it independently of the middle arm 18 and the first outer arm 20. The second and third threaded shafts 26 and 32 of this embodiment can engage the threaded openings 28 and 30 disposed in the first and second outer arms 20 and 22 to facilitate movement of the first and second outer arms 20 and 22. All of the threaded shafts 24, 26 and 32 described herein can include a driving head 34 to allow a user to be able to turn the threaded shafts 24, 26 and 32. The driving head 34 can be any type so that a driving tool can be used to turn the threaded shafts. For example, the driving heads 34 can be a Philips pattern, a flat head pattern, a star pattern, a hex pattern, a TORX pattern, and the like. The axis of rotation for the driving tool used to rotate the threaded shafts 24, 26 or 32 is in line with the axis that the threaded shafts 24, 26 or 32 rotates about, respectively.

The retractor body 16 can have a first slot 36 and a second slot 38 disposed therein for receiving the first and second outer arms 20 and 22, respectively. One end of the first outer arm 20 is slidably disposed in the first slot 36 and one end of the second outer arm 22 is slidably disposed in the second slot 38. The middle arm 18 is supported by the retractor body 16 between the first and second slots 20 and 22. Each arm (middle, first outer, and second outer) 18, 20 and 22 can include a secure opening 40 therein that can be used as a table-arm fixation point to secure the medical retractor 14 in a fixed position relative to an operating table. Each secure opening 40 can have a locking device 42 around it that can engage with the table-arm to secure the position of the medical retractor 14 relative to the operating table. For example, the secure opening 40 can contain a thread, groove, circular tooth pattern, and the like. The secure opening 40 may be an insert that is permanently fixated to the respective arm.

A cross-bar 400 can be included with the medical retractor assembly 10 to be selectively securable to the medical retractor 14 to permit additional instruments to be used with the medical retractor assembly 10. The cross-bar 400 can include a slot 402 disposed therein for allowing the cross-bar 400 to be moveable with respect to the medical retractor 14 and to permit attachment means to secure the cross-bar 400 to the medical retractor 14. The attachment means can include at least one ball-tip screw 404 (or cylindrical-tip screw) wherein the shaft 406 can extend through the slot 402 and threaded into holes 408 disposed in the ends 410 of the first and second outer arms 20 and 22. The heads 412 of the ball-tip screws 404 are sized such that they cannot pass through standard areas of the slot 402 of the cross-bar 400. The slot 402 can include wider portions 414 disposed therein that permit the heads 412 of the ball-tip screws 404 to pass therethrough to permit the cross-bar 400 to be decoupled from the medical retractor 14 without removing the ball-tip screws 404 from the arms 20 and 22. The cross-bar 400 can also include a blade retainer 416, such as a hook, attached to the back side of the cross-bar 400 and a retention device 418, such as a screw, extending through the cross-bar 400 and into the space inside the blade retainer 416. The retention device 418 can be used to engage an instrument that extends through a space in the blade retainer 416 and secure the instrument in a desired position.

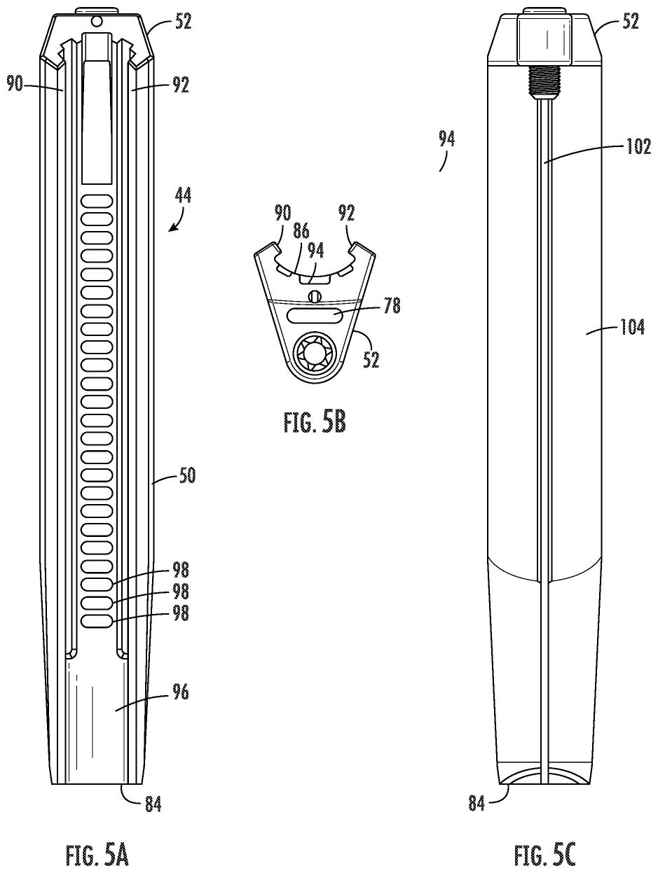

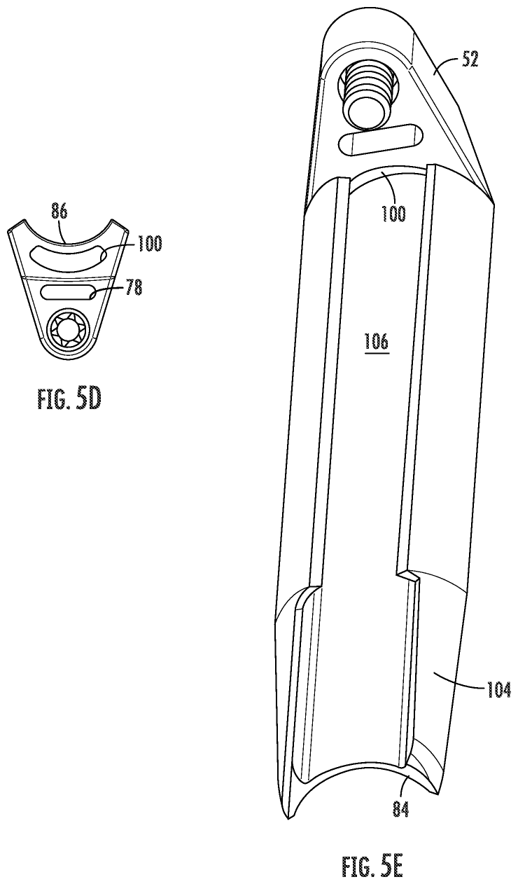

Each arm (middle, first outer, and second outer) 18, 20 and 22 can support a retractor blade. A middle retractor blade 44 extends from the middle arm 18 in a generally perpendicular direction from the plane shared by the arms 18, 20 and 22. Similarly, a first outer retractor blade 46 and a second outer retractor blade 48 extends from the first outer arm 20 and the second outer arm 22, respectively, in a direction that is generally perpendicular to the plane shared by the arms 18, 20 and 22. Referring now to FIGS. 5A-5C, each retractor blade 44, 46 and 48 can be identical and removably attached to its respective arm in any manner known in the art. The retractor blades 44, 46 and 48 each have a blade portion 50 and an attachment end 52 that extends from the blade portion 50. The retractor blades 44, 46 and 48 can be attached to their respective arms of the medical retractor 14 before or after insertion of one or more of the retractor blades 44, 46 and 48 into the patient on which surgery is being performed. A temporary handle (not shown) can be attached to the attachment end 52 of the retractor blade 44, 46 or 48 to insert a first, second, or third retractor blade prior to inserting and attaching the respective retractor arms 18, 20 and 22 to the retractor blade 44, 46 and/or 48.

In one embodiment of the present disclosure and shown in more detail in FIGS. 3G and 3H, each retractor blade 44, 46 and 48 can be rotatably supported by each respective arm 18, 20 and 22 and can rotate independently with each respective arm 18, 20 and 22. In this embodiment, the axis of rotation for the first outer and second outer blade 46 and 48 for angulation or toeing is not parallel to an axis generally defined as an axis in the direction of the first or second outer arm 20 and 22 as it extends from the body 16. In an exemplary embodiment, each arm includes a cutout portion 54 that has a base 56, a first sidewall 58, a second sidewall 60 and a back wall 62. A rotatable platform 64 can be positioned in the cutout portion 54 of each arm 18, 20 and 22 where the rotatable platform 64 can rotate around a pin 66 that extends through a hole 68 in the rotatable platform 64 and into holes 70 in the first and second sidewalls 58 and 60 of the cutout portion 54. In another embodiment, the rotatable platform 64 can have pins extending therefrom that are inserted in the holes 70 in the first and second sidewalls 58 and 60 of the cutout portion 54 to facilitate rotation of the rotatable platform 64. The rotatable platform 64 includes a back 72 on one side of the rotation axis and a front 74 on the opposite side of the rotation axis. The attachment end 52 of each retractor blade 44, 46 and 48 can be secured to the rotatable platform 64 that is rotatably disposed in each cutout portion 54 of each arm 18, 20 and 22, respectively. The attachment end 52 of each retractor blade 44, 46 and 48 can be secured to the rotatable platform 64 via any means known in the art, such as with a screw. In a further embodiment, and to further secure the relationship between each retractor blade 44, 46 and 48 and each respective rotatable platform 64, each rotatable platform 64 can have a flange 76 that extends upward and can extend into a slot 78 disposed in the attachment end 52 of the respective retractor blade 44, 46 and 48.

To facilitate rotation of the rotatable platform 64 in the cutout portion 54 of each arm 18, 20 and 22, and thus each retractor blade 44, 46 and 48 attached to each rotatable platform 64, each rotatable platform 64 can have a threaded opening 80 therein to receive an adjustment screw 82. The threaded opening 80 can be generally perpendicular and offset from the rotation axis of the rotatable platform 64 so that rotation of the rotatable platform 64 is correct for proper operation of the medical retractor 14. The adjustment screw 82 can thread into the threaded opening 80 in the back 72 of the rotatable platform 64. When turned a specific amount, the adjustment screw 82 can extend through the threaded opening 80 in the rotatable platform 64 and pushes against the base portion 56 of the cutout portion 54 of each arm 18, 20 and 22. As the adjustment screw 82 pushes against the base portion 56, it causes the back 72 of the rotatable platform 64 to move away from the base portion 56 of the cutout portion 54 and the front 74 of the rotatable platform 64 to move toward the base portion 56. The rotation of each rotatable platform 64 is translated to the rotation of each retractor blade 44, 46 and 48, which causes a distal tip end 84 of each retractor blade 44, 46 and 48 to rotate outward and away from the distal tip ends 84 of the other respective retractor blades 44, 46 and 48. In this embodiment, the resistance on the back surface 104 of the respective retractor blade 44, 46, and 48 transfers to the rotatable platform and subsequently the adjustment screw 82 to allow continuous contact between the base portion 56 and the adjustment screw 82.

Referring again to FIGS. 5A-5C, each retractor blade 44, 46 and 48 includes a shim channel 86 that can run a portion of the entire length of the blade portion 50, a portion of the entire length of the blade portion 50 of each retractor blade 44, 46 and 48, or the entire length of the blade portion 50. The inside surface of each retractor blade 44, 46 and 48 creates the base 88 of the shim channel 86. In one embodiment, the base 88 of the shim channel 86 is generally rounded, which creates a concave cross-section of the shim channel 86 from inside each retractor blade 44, 46 and 48. In addition to the inside of each retractor blade 44, 46 and 48, each shim channel 86 is defined by a first flange 90 and a second flange 92. The first and second flanges 90 and 92 can be angled or curved back towards the center of the shim channel 86 to allow devices, or parts of devices, to be slid down into and through the shim channel 86 and only allow movement of the devices in the direction of the length of the blade portion 50 of each retractor blade 44, 46 and 48. Thus, regardless of position along the shim channel 86, the cross-sectional area (i.e., cross-section perpendicular to the length of the blade portion 50 of the retractor blade 44, 46 or 48) of the shim channel 86 will not change. In an alternate embodiment the cross-section of the shim channel 86, perpendicular to the length of the blade portion 50, can have a specific shape. This cross-section shape may be arc shaped, cylindrical, or some other shape that allows mechanical engagement of a tool. In one embodiment, the blade portion 50 of each retractor blade 44, 46 and 48 can taper towards the end that is extended into the surgical site. It should be understood and appreciated that the cross-sectional area of the shim channel 86 would still not change even as the blade portion 50 is tapered.

In a further embodiment of the present disclosure, in addition to the shim channel 86, the inside of the blade portion 50 (or the base portion 88 of the shim channel 86) can include a second channel 94 or second and third channel 94 and 96 that is disposed along the length or a portion of the length of the blade portion 50 of each retractor blade 44, 46 and 48. Disposed inside the base portion 88 of the shim channel 86 can be a series of blind slots 98 that can engage and secure the placement of certain tools that can be used with the medical retractor 14.

In yet another embodiment, the attachment end 52 of each retractor blade 44, 46 and 48 can have an opening 100 therein that is in general alignment with a cylindrical-shaped channel 102 that runs along a back side 104 or outside surface of the respective blade portion 50 of each retractor blade 44, 46 and 48. The channel 102 does not communicate with the inside surface of each of each retractor blade 44, 46 and 48. The cylindrical-shaped channel 102 can be fully enclosed or only partially enclosed. The opening 100 in the attachment end 52 of each retractor blade 44, 46 and and the cylindrical-shaped channel 102 cooperate to permit neuromonitoring probes or fixation tools, such as a wire, to be extended down to the surgical site to monitor various physiological characteristics desired or provide fixation of the medical retractor 14 to the spine 12. In an even further embodiment and shown in more detail in FIGS. 5D and 5E, each opening 100 in the attachment end 52 of each retractor blade 44, 46 and 48 can be arc-shaped and in general alignment with a second shim channel 106 disposed on the back side 104 or outside surface of the respective blade portion 50 of the retractor blade 44, 46 and 48. The second shim channel 106 can be disposed in the back side 104 of each retractor blade 44, 46 and 48 such that a portion of the blade portion 50 can partially surround the medical tool slid down the second shim channel 106. Alternatively, the second shim channel 106 can be disposed in the back side 104 of each retractor blade 44, 46 and 48 such that the blade portion 50 fully surrounds or encloses the medical tool slid down the shim channel 86. It should be understood and appreciated that each of the retractor blades 44, 46 and 48 herein can have any, or all, of the characteristics described herein.

The medical retractor 14 can have a fully open position and a closed position. When the medical retractor 14 is in the closed position, the first flange 90a of the middle retractor blade 44 attached to the middle arm 18 contacts the second flange 92b of the retractor blade 46 attached to the first outer arm 20, the first flange 90b of the retractor blade 46 attached to the first outer arm 20 contacts the second flange 92c of the retractor blade 48 attached to the second outer arm 22 and the first flange 90c of the retractor blade 48 attached to the second outer arm 22 contacts the second flange 92a of the middle retractor blade 44 attached to the middle arm 18. The arms 18, 20 and 22 can be manipulated to create a desired openness and orientation of the retractor blades 44, 46 and 48 of the medical retractor 14 between the closed position and the fully open position. In the fully open position, the middle arm 18 is retracted back towards the retractor body 16 as far as it can be moved, and the first and second outer arms 20 and 22 are moved as far in their respective slots 36 and 38 in the retractor body 16, away from the middle arm 18. When the medical retractor 14 is in the fully closed position, the three blade portions 50 of the three retractor blades 44, 46 and 48 have an outer surface profile, created by the back sides 104 of each blade portion 50 of the retractor blades 44, 46 and 48, wherein the outer surface profile of the three blades 44, 46 and 48 form a scalloped, non-concentric shape and the length of all three blades 44, 46 and 48 is the same such that the end of the blade portion 50 of all three retractor blades 44, 46 and 48 is the same. In an alternate embodiment one or two of the retractor blades may be longer or shorter than the other respective blades so as to further fit the anatomy of the surgical site. Additionally, when the medical retractor 14 is in the fully closed position, the three blade portions 50 of the three retractor blades 44, 46 and 48 have an inner surface profile, created by the inside surface formed by the base of the shim channel 86 and the various first and second flanges 90 and 92 of the retractor blades 44, 46 and 48. When cross sectioned along the length of the three blades in the closed position, the base 88 of the shim channel 86 surfaces of all three blades 44, 46 and 48 form a non-continuous circular and concentric profile, and the inside surface of the various first and second flanges 90 and 92 on each blade form a non-continuous circular and concentric profile of a smaller diameter than the base shim channel circle.

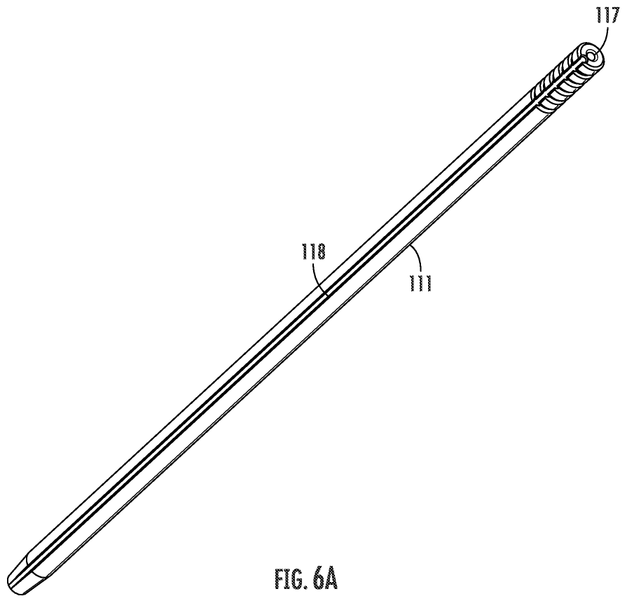

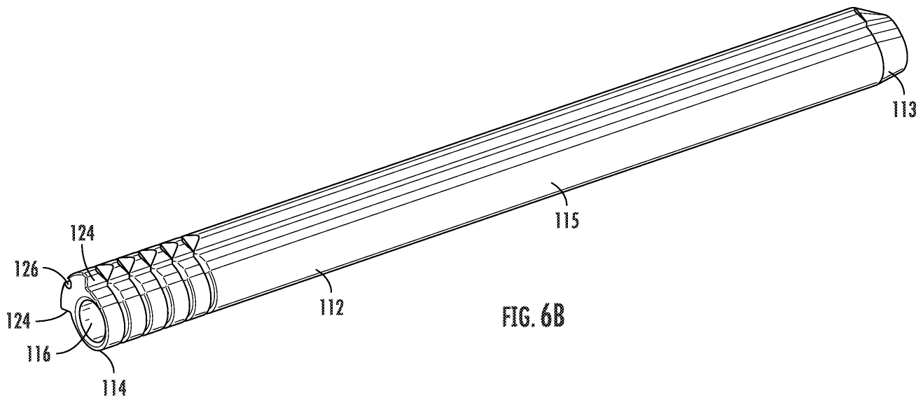

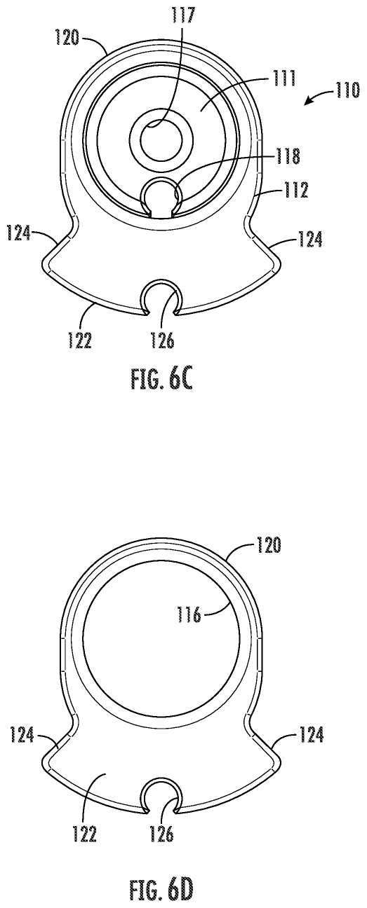

In a further embodiment of the present disclosure and shown in more detail in FIGS. 6A-6E, the medical retractor assembly 10 can also include a first shim feature dilator 110 made up of two parts: an inner member 111 that is slidably disposed in an outer member 112. The first dilator 110 can have a first end 113 of the outer member 112 that is tapered to engage portions of the tissue in the surgical site, a second end 114 of the outer member 112 the user of the medical retractor assembly can grasp to manipulate the outer member 112 of the first dilator 110, and a body 115 that extends between the first end 113 and the second end 114 of the outer member 112. The first dilator 110 can also have a hole 116 that runs down through the length thereof for the inner member 111 to be able to extend into and through. The inner member 111 of the first dilator 110 can have a first end 111a that is tapered to engage portions of the surgical site, a second end 111b to provide the user something to grasp when manipulating the inner member 111, and a body 111c that extends between the first end 111a and the second end 111b of the inner member 111. The inner member 111 can also include a central hole 117 disposed therethrough where a guide wire can be extended therethrough. The inner member 111 can also include a channel 118 disposed on the outside of the inner member 111 that a guide wire or neuromonitoring probe can be slidably disposed therethrough. A cross-section of the inner member 111 is primarily circular shaped. In an alternate embodiment the inner member 111 and outer member 112 can be combined to form a singular dilator 110.

A cross-section of the first dilator 110, perpendicular to the length of the first dilator 110, can have a specific shape. The specific shape of the cross-section of the first dilator 110 includes a round portion 120 (or an elongated round portion) and an arc-shaped portion 122 attached to the round portion 120. The arc-shaped portion 122 of the first dilator 110 is sized and shaped such that is can slidably engage along the length of any of the shim channels 86 of the retractor blades 44, 46 and 48. The flanges 90 and 92, which extend from one of the blade portions 50 of the retractor blades 44, 46 and 48 that create one of the shim channels 86, engage with the ends 124 of the arc portion 122 when the first dilator 110 is slid down through the medical retractor 14 or alternatively when the medical retractor 14 is slid down over the first dilator 110. In a further embodiment, the first dilator 110 can also include an outer channel 126 that is either fully enclosed or partially enclosed that runs down the outside of the arc-shaped portion 122 of the first dilator 110. The outer channel 126 can be used to extend a surgical tool such as a neuromonitoring probe or fixation wire down to the surgical site.

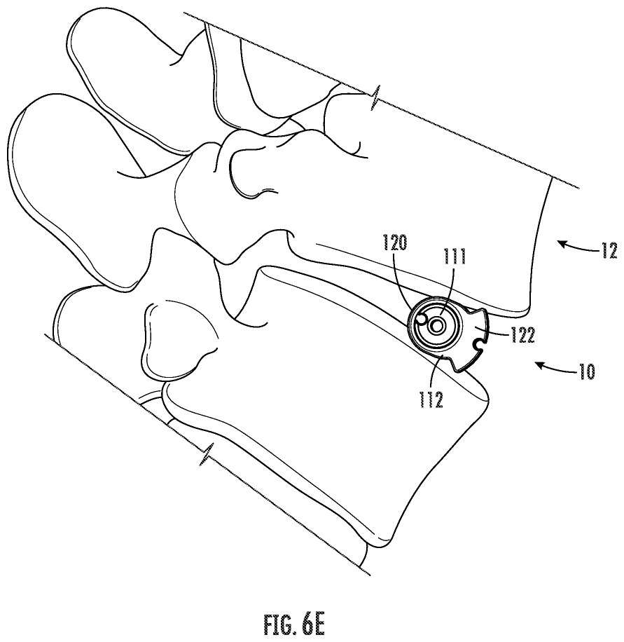

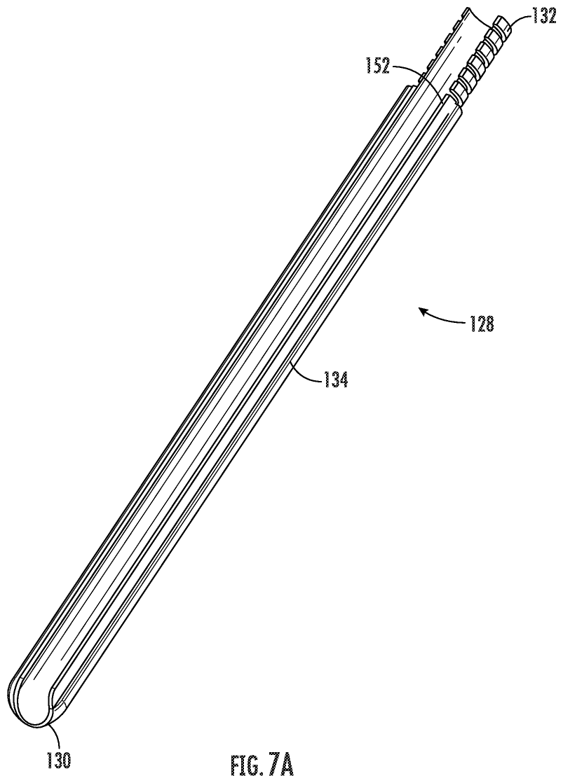

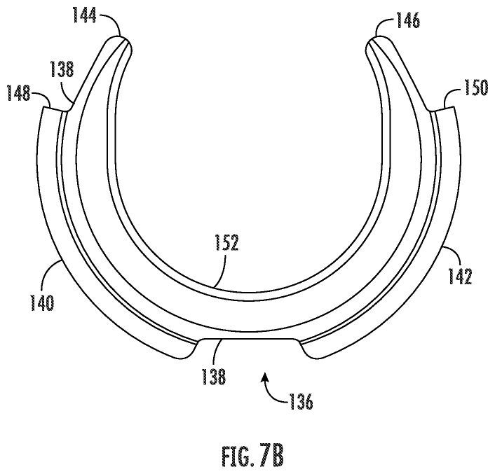

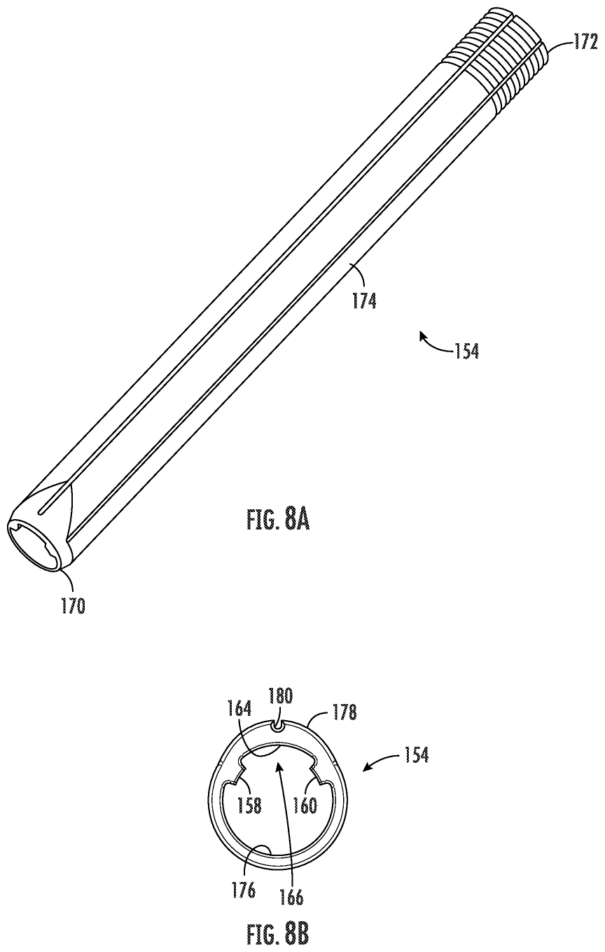

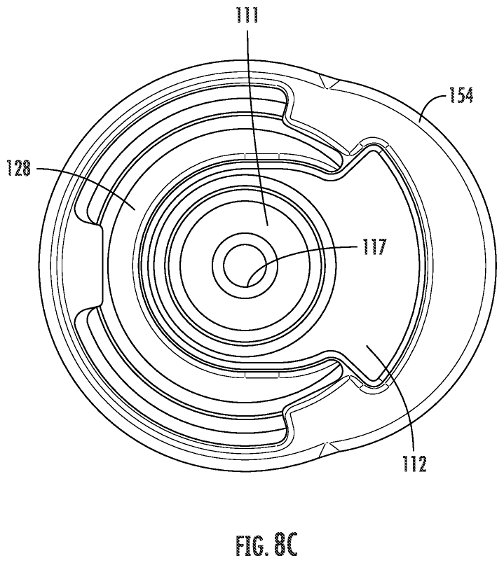

Referring now to FIGS. 7A-7B, the medical retractor assembly 10 can also include a second dilator 128 that can have a first end 130 that can be tapered to engage portions of the tissue in the surgical site, a second end 132 the user of the medical retractor assembly 10 can grasp to manipulate the second dilator 128, and a body 134 that extends between the first end 130 and the second end 132. In one embodiment, the second dilator 128 is sized and shaped such that the first dilator 110 and the second dilator 128 can be married up and fill in the space between the inside surfaces of the retractor blades 44, 46 and 48 when the medical retractor 14 is in the fully closed position. A cross-section of the body 134 of the second dilator 128, perpendicular to the length of the second dilator 128, can have a specific shape. The specific shape of the cross-section of the second dilator 128 is roughly a crescent-moon shape. In this embodiment, the inside dimensions of the crescent-moon shape is such that it can receive the round portion 120 of the first dilator 110 and the arc portion 122 of the first dilator 110 extends radially beyond the outer surface of the crescent-moon shape of the second dilator 128 such that the ends 124 of the arc portion 122 of the first dilator 110 are accessible. The outer diameter of the crescent-moon shape of the second dilator 128 abuts the shim flanges 90 and 92 of the three retractor blades 44, 46 and 48 when they are in the closed position. The arc portion 122 of the first dilator 110 abuts the base portion 88 of the shim channel 86 in a retractor blade 44, 46 or 48. In yet another embodiment, the cross-sectional crescent-moon shape can include a depression 136 therein that is consistent with a channel that runs the length of the second dilator 128 on an outer side 138 of the second dilator 128. The depression 136 (and thus channel) can receive the flanges 90 and 92 of two adjacent retractor blades 44, 46 or 48. A first lobe 140, similar to the arc-portion 122 of the first dilator, can be disposed on the outer side 138 of the second dilator 128 that can be slidably engaged within the shim channel 86 of the retractor blades 44, 46 or 48. A second lobe 142, similar to the arc-portion 122 of the first dilator, can be disposed on the outer side 138 of the second dilator 128 that can be slidably engaged within the shim channel 86 of the retractor blades 44, 46 or 48. The second lobe 142 is separated from the first lobe 140 by the depression 136. The lobes 140 and 142 are slidably disposable in the shim channels 86 disposed in two adjacent retractor blades 44, 46 or 48 as seen in FIG. 8D. The cross-sectional crescent-moon shape can also include a first extended portion 144 extending beyond the first lobe 140 and a second extended portion 146 extending beyond the second lobe 142. The length of the first and second extended portions 144 and 146 are roughly equal to the width of an adjacent set of flanges 90 and 92 on adjacent retractor blades 44, 46 or 48. In this embodiment, the round portion 120 of the first dilator 110 can engage along the length of the inside 152 of the crescent-moon portion of the second dilator 128. The outer profile created by the combined first and second dilators 110 and 128 can match the internal profile of a fully enclosed third dilator 154 in certain embodiments. The first extended portion 144 and the first lobe 140 forming a first ledge 148 on the second dilator 128 receives a first flange 158 on the inside profile of a third dilator 154 (shown in FIGS. 8A-8C). The first flange 158 of the inside profile of a third dilator 154 abuts the first ledge portion 148 of the second dilator 128 and a second flange 160 of the inside profile of the third dilator 154 abuts a second ledge portion 150 of the second dilator 128, created by the second extended portion 146 and the second lobe 142, and the arc portion 122 of the first dilator 110 abuts a base portion 164 of a shim channel 166 in the third dilator 154. In a further embodiment, the second dilator 128 can also include an outer channel (not shown) that is either fully enclosed or partially enclosed that runs down the outside diameter of the second dilator 128. The outer channel can be used to extend a surgical tool such as a neuromonitoring probe or fixation wire down to the surgical site. The arc portion 122 of the first dilator 110 and the first and second lobes 140 and 142 of the second dilator 128, when engaged with the shim channels 86 disposed in the three blades 42, 44 and 46, respectively, prevent the blades 42, 44 or 46 from opening or angulating as the medical retractor 14 is moved to the surgical site via the first and second dilators 110 and 128. The outer surfaces of the arc portion 122 of the first dilator 110 and the first and second lobes 140 and 142 of the second dilator 128 form a non-continuous circular profile concentric about the guide wire hole 117 in the first dilator 110.

Furthermore, the third dilator 154 of the medical retractor assembly 10 can also have a first end 170 that can be tapered to engage portions of the tissue in the surgical site, a second end 172 the user of the medical retractor assembly 10 can grasp to manipulate the third dilator 154, and a body 174 that extends between the first end 170 and the second end 172. The third dilator 154 is comprised of an inner wall 176 and outer wall 178 and is configured on the inside to permit the first and second dilators 110 and 128 to be extended into and through the third dilator 154. In one embodiment, the internal profile of the third dilator 154 would be similar to the internal profile of the retractor blades 44, 46 and 48 when the medical retractor 14 is in the fully closed position as shown in FIG. 8D. Thus, the third dilator 154 would include at least one shim channel 166 therein. In an alternate embodiment, the internal profile of the third dilator 154 would only include one shim channel 166 and the shim flanges 158 and 160 between the first and second outer retractor blades 44 and 46 would be removed such that the inner diameter of the third dilator 154 is substantially the same as the diameter of the base portion 164 of the shim channels 166, thus having the same inner cross-sectional profile as the retractor blades 44, 46 and 48 when they are in the fully closed position. Further, the third dilator 154 has an outer profile that is partially scalloped and non-concentric. The third dilator 154 can also include an outer channel 180 that is either fully enclosed or partially enclosed that runs the length of the third dilator 154 where a surgical tool such as a neuromonitoring probe could be extended down to the surgical site.

One unique aspect of the medical retractor assembly 10 is the series of concentric circles created by the first dilator 110, the second dilator 128 and the retractor blades 44, 46 and 48 around the central hole 117 of the inner member 111. The first concentric circle is defined by the inner member 111 of the first dilator 110. The second concentric circle is defined by the round portion 120 of the first dilator 110. The third concentric circle is defined by the outer side 138 of the second dilator 128 and the inside surface of the respective adjacent first and second flanges 90 and 92. The fourth concentric circle is defined by the outside of the lobes 140 and 142 of the second dilator 128 and the inside of the shim channels 86 of the retractor blades 44, 46 and 48.

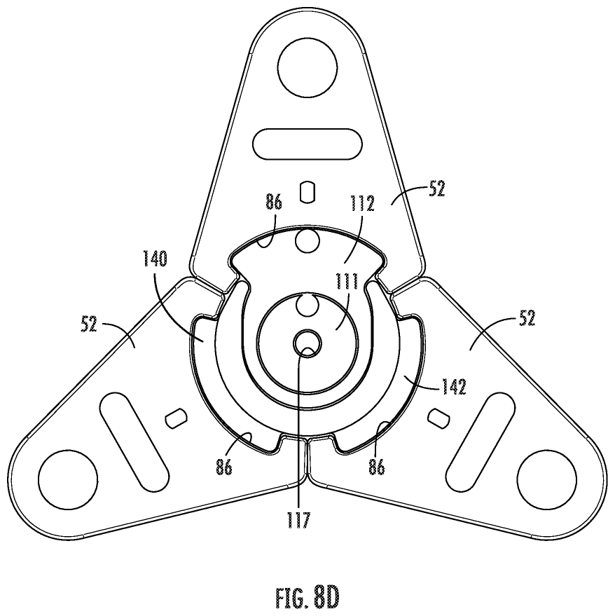

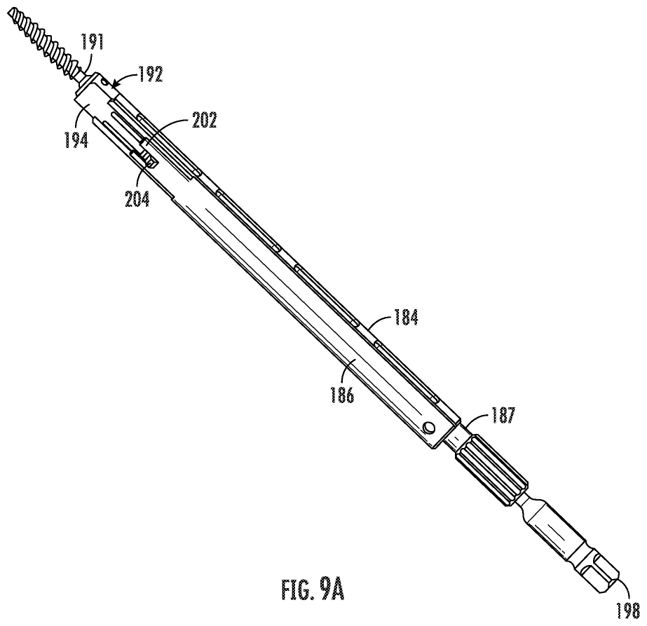

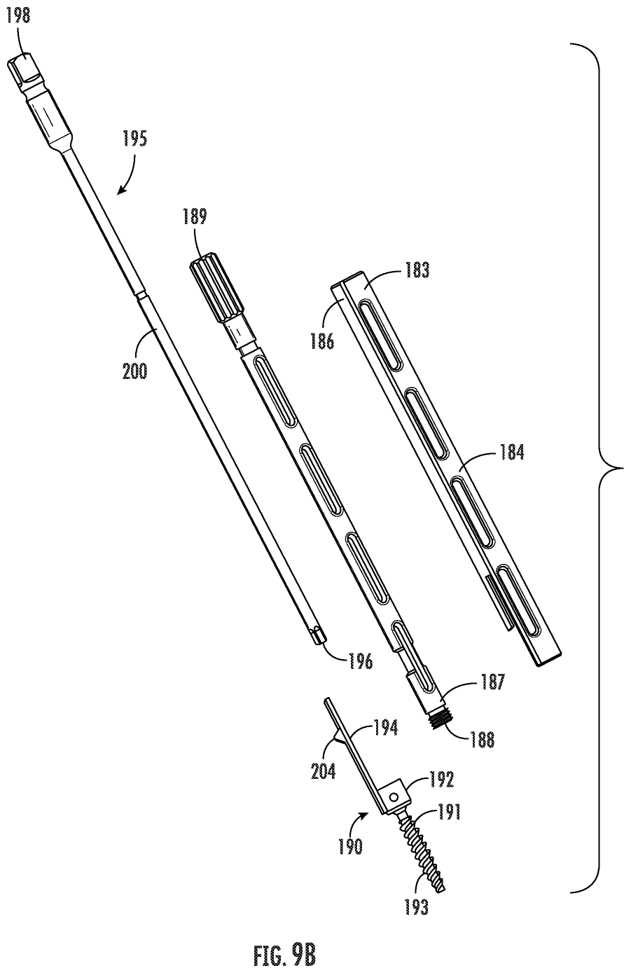

Referring now to FIGS. 9A-9B, the medical retractor assembly 10 can also include a threaded fixation apparatus 182 that has an outer body 183 that includes a round body component 184 for working with other components (e.g., the second dilator 128) of the medical retractor 14 and an shim component 186 (the shim components 186 having an arc-shaped cross-sectional shape) to facilitate cooperation with the medical retractor 14. The threaded fixation apparatus 182 can also include an inner body 187 that can be inserted through the outer body 183. The inner body 187 can have a first end 188 for providing a user a place to manipulate the inner body 187 and the threaded fixation apparatus 182 and a second end 189 that can be selectively secured (such as via threads) to a fixation pin support member 190 that supports a threaded fixation pin 191. The fixation pin support member 190 can include a cup portion 192 having a hole (not shown) therein to permit a threaded portion 193 of the threaded fixation pin 191 to extend therethrough to engage a surgical site. The fixation pin support member 190 can also include a slat member 194 that extends from the cup portion 192 to engage with the outer body 183 of the threaded fixation apparatus 182. The threaded fixation apparatus 182 can also include a driving apparatus 195 that has a drive end 196 for engaging a polyaxial head (not shown) of the threaded fixation pin 191 and a handle end 198 for providing a means for manipulating the driving apparatus 195 and rotatably advancing the threaded fixation pin 191. The driving apparatus 195 can have a body portion 200 that can extend through the inner body 187 and the outer body 183 of the threaded fixation apparatus 182 and between the drive end 196 and the handle end 198. The shim component 186 of the outer body 183 can include a pair of flanges 202 that extend axially therefrom that can engage a protruded portion 204 that extends outward from the slat member 194 of the fixation pin support member 190. The drive end 196 and receiving feature (not shown) of the polyaxial head in the threaded fixation pin 191 can be a Philips pattern, a flat head pattern, a star pattern, a hex pattern, a TORX pattern, and the like. The protruded portion 204 can also engage with the inside channels and blind slots of the retractor blades 44, 46 and 48. The polyaxial head can be attached to the threaded portion 193 to prevent the threaded fixation pin 191 from sliding through the hole of the cup portion 192 of the fixation pin support member 190. Additionally, a hole (not shown) is disposed in the polyaxial head, the threaded portion 193 of the threaded fixation pin 191 and the driving apparatus 195 to permit a guide wire to extend all the way through the threaded fixation apparatus 182.

The outer surface, cross-sectional shape of the outer body 183 and the shim component 186 match the outer surface, cross-sectional shape of the first dilator 110 described herein. The shim component 186 is shaped such that it can slide into and through the shim channel 86 of any of the retractor blades 44, 46 and 48 described herein. Alternatively, the fixation apparatus 182 can be delivered through the second and third dilators 128 and 154. The third dilator 154 can be removed and the medical retractor 14 can, with the blades in the fully closed position, be delivered over the fixation apparatus 182 such that the shim channel 86 of any of the retractor blades 44, 46 and 48 can engage with the arc shaped shim component 186 of the fixation apparatus 182.

The protruded portion 204 extending from the slat member 194 of the fixation pin support member 190 is sized to engage the blind slots 98 of the series of blind slots 98 disposed in the secondary channel 94 of the blade portion 50 of the retractor blades 44, 46 or 48. The flexibility of the slat member 194 permits a user to force the threaded fixation apparatus 182 past the blind slots 98 to position the threaded fixation apparatus 182 at a desired position relative to the medical retractor 14 or force the medical retractor 14 to a desired position relative to the threaded fixation apparatus 182.

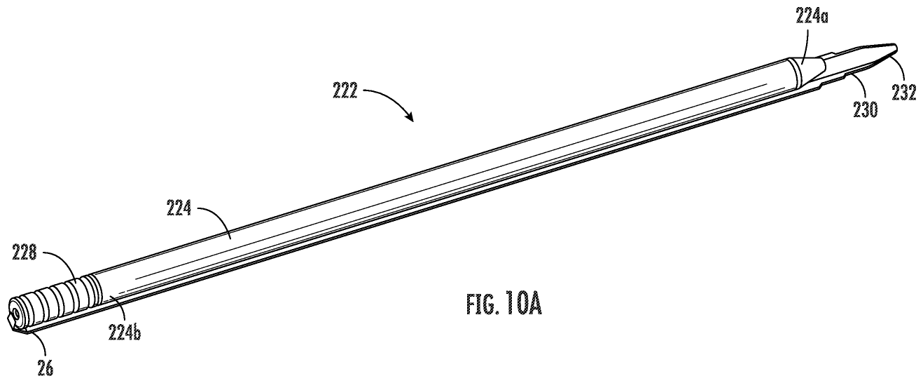

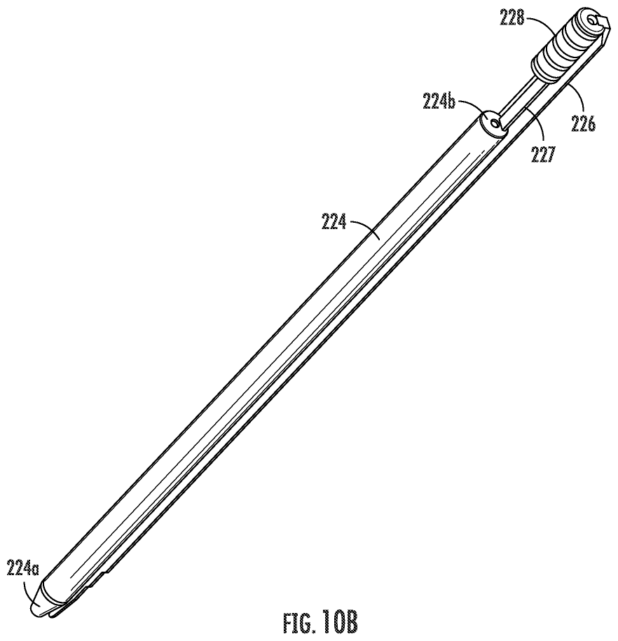

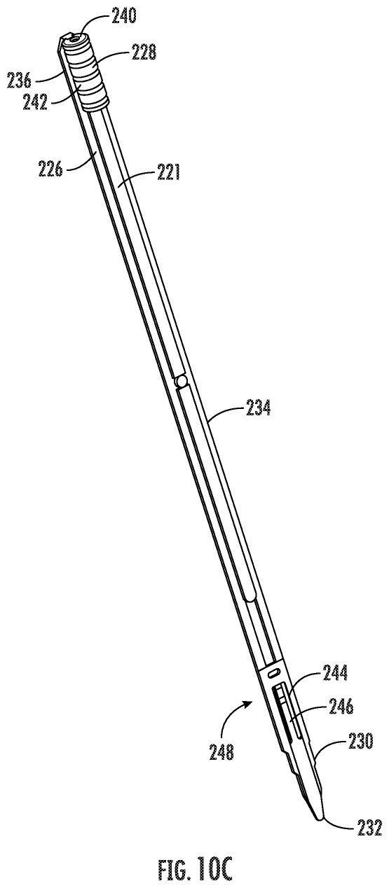

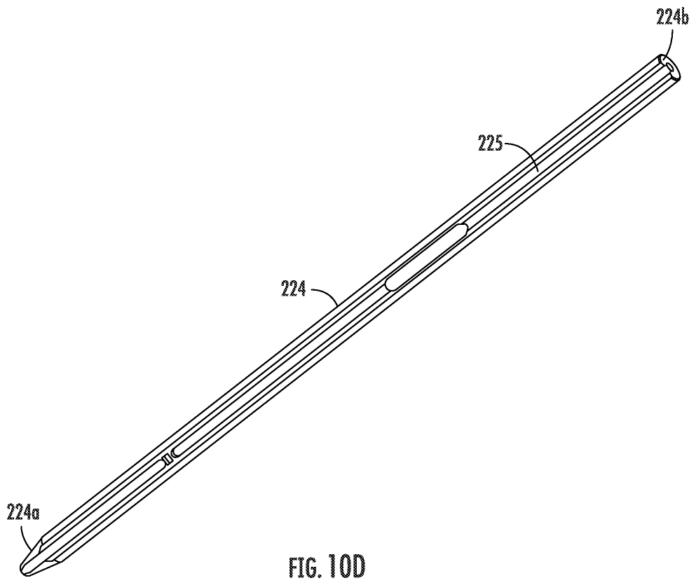

Referring now to FIGS. 10A-10D, the medical retractor assembly 10 can also include a shim fixation apparatus 222 that has a round body component 224 for accessing the surgical site, a shim component 226 to facilitate cooperation with the medical retractor 14 and engage with parts of the surgical site, and an optional shim extension 228 that can be used to control the shim component 226. The round body component 224 includes a first end 224a with a rounded or tapered shape which helps advance through tissue at the surgical site and a second end 224b that allows the user of the medical retractor 14 to control the shim fixation apparatus 222. The body component 224 has a channel 225 disposed along the length of the body component 224 that is in alignment and matingly engages a ridge 227 that runs along the length of a portion of the shim component 226. In some embodiments, the channel 225 and ridge 227 can be designed such that the ridge 227 has to be slid into the channel 225 for the body component 224 and the shim component 226 to make up the shim fixation apparatus 222. The shim component 226 is similar to the shim component 186 of the threaded fixation apparatus 182 described herein. The shim component 226 can have a first end 230 that can be a tapered blade 232 that can be used to engage disc space between vertebrae, a second end 236 that contains a feature that engages with the inside channels 86 and blind slots 98 of the retractor blades 44, 46 and 48 and a shim body 234 that extends from the tapered blade 232 of the first end 230 to the second end 236. Optionally, the second end 236 of the shim component 226 contains a feature that engages with the shim extension 228. The shim extension 228 contains a first end 238 that engages with the second end 236 of the shim component 226 and a second end 240 that can be manipulated by the user of the medical retractor assembly 10. The shim extension 228 extends from the second end 236 of the shim component 226 to the second end 224b of the round body component 224. The second end 240 of the optional shim extension 228 can include a cylindrical portion 242 attached thereto with an opening (not shown) therein to permit the guide wire to be extended therethrough. The cross-sectional shape of the second end 240 of the shim extension 228 matches the cross-sectional shape of the first dilator 110 described herein. The shim body 234 of the shim extension 228 and shim component 226, as well as parts of the first and second end 238 and 240, are shaped such that they can slide into and through the shim channel 86 of any of the retractor blades 44, 46 and 48 described herein. The shim component 226 can also include an opening 244 therein and a slat 246 extending up into the opening 244 with a flange 248 extending therefrom. The slat 246 in the opening 244 leaves space on the sides of the slat 246 and a specific sized space at the end of the slat 246 which allows the slat 246 to be somewhat flexible in the opening 244 in the shim body 234. The flange 248 extending from the end of the slat 246 is sized to engage the blind slots 98 of the series of blind slots 98 disposed in the secondary channel 94 of the blade portion 50 of the retractor blade 44, 46 or 48. The flexibility of the slat 246 permits a user to force the shim fixation apparatus 222 past the blind slots 98 to position the shim fixation apparatus 222 at a desired position relative to the medical retractor 14, or force the medical retractor 14 to a desired position relative to the shim fixation apparatus 222. In an alternate embodiment, the second end of the shim component 236 may contain a pin feature (not shown) that slides along the second and/or third channels 94 and/or 96 contained within the base portion 88 of the shim channel 86 disposed along the length or a portion of the length of the blade portion 50 of each retractor blade 44, 46, and 48.

The body component 224 of the shim fixation apparatus 222 is similar to the body component 184 described in the threaded fixation apparatus 182 but is used a little differently. The shim component 226 and optional shim extension 228 of the shim fixation apparatus 222 can be slidably moved relative to the round body component 224 into two general states. The first state is where the tip of the tapered blade 232 of the shim component 226 is flush with the tip of the first end of the round body component 224 to facilitate moving the shim fixation apparatus 222 through tissue more easily. Once the shim fixation apparatus 222 in its first state has been moved to the desired position within the surgical site, the tapered blade portion 232 of the shim component 226 of the shim fixation apparatus 222 can be advanced further beyond the first end of the round body component 224 into the surgical site (second state). Subsequently, the second end of the round body component 224 will advance closer to the optional cylindrical portion 242 of the shim extension 228. The body component 224 can be generally cylindrical in shape and, together with the shim body 234 of the shim extension 228 and shim component 226, would create a cross-sectional shape that is similar to the first dilator 110 described herein. The body component 224 can be selectively removed from the shim fixation apparatus 222 by the user when desired. In one embodiment, the body component 224 is frictionally or mechanically engaged with the optional shim extension 228 and/or shim component 226 of the shim fixation apparatus 222. Similar to the second end 240 of the shim extension 228, the body component 224 can have a hole running through it so that a guide wire 260 can be extended therethrough.

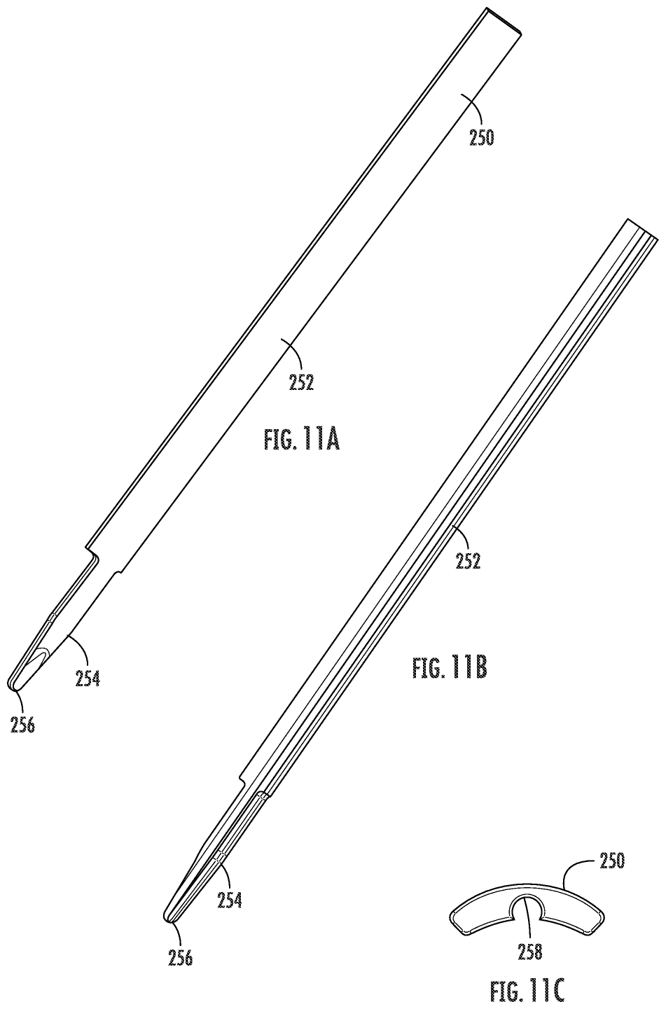

Referring now to FIGS. 11A-11D, the medical retractor assembly 10 can also include a shim tool 250 that includes a shim body 252 for facilitating use of the shim tool 250 in the shim channel 86 of the retractor blades 44, 46 or 48 of the medical retractor 14 and a shim head 254 for engaging the desired area of the surgical site, such as the disc space between two adjacent vertebrae. FIG. 11D shows the shim tool 250 in use with the retractor blades 44, 46 or 48 shown in FIGS. 5D and 5E. A cross-section of the shim body 252 of the shim tool 250, perpendicular to the length of the shim tool 250, can have a specific shape to engage with the shape of the shim channel 86 of the retractor blades 44, 46 and 48 as shown in FIG. 11D. This cross-section shape may be arc-shaped, cylindrical, or some other shape that allows mechanical engagement with the shim channel 86 on the retractor blade 44, 46 and 48. The shim head 254 can be a flattened, triangular shape with a tapered tip 256 to allow the shim head 254 to engage a smaller space due to the tip 256 of the shim head 254 being smaller than the shim head 254 at its widest point. The shim body 252 and shim head 254 can have a fully enclosed or partially enclosed circular channel 258 disposed therein for permitting a guide wire to be extended therethrough, or for permitting the shim tool 250 to be slid down over the guide wire.

Various methods can be undertaken with the devices and apparatuses described herein. In one embodiment, a pedicle of the inferior/caudal vertebral body of the disc level being treated is located via a TLIF surgical approach and a guide wire 260 (shown in FIG. 1) can be inserted into the pedicle. The first dilator 110 can be advanced down to the desired location of the surgical site (could be bone) over the guide wire 260. The second dilator 128 can be advanced down to the desired location of the surgical site over the first dilator 110 and the guide wire 260. The third dilator 154 can be advanced down to the desired location of the surgical site over the second dilator 128, the first dilator 110 and the guide wire 260. After the first, second and third dilators 110, 128 and 154 are advanced down to the desired location of the surgical site over the guide wire 260, the first dilator 110 can be removed. The threaded fixation apparatus 182 described herein can be advanced around the guide wire 260 and inside the second and third dilators 110 and 128. The threaded fixation pin 191 can be screwed into the pedicle wherein the polyaxial head and the ring portion of the first end of the shim component 186 of the threaded fixation apparatus 182 secure the shim component 186 in a desired location. In one embodiment, the threaded fixation pin 191, without a polyaxial head, can be secured to a vertebral, or some other area of the surgical site.

Once the threaded fixation pin 191 is secured to the pedicle, the third dilator 154 can be withdrawn and the medical retractor 14, typically in the closed position, can be advanced down over the second dilator 128 and the threaded fixation apparatus 182. Any of the shim channels 86 of the retractor blades 44, 46 and 48 from the medical retractor 14 can be selected to engage the shim component 186 of the threaded fixation apparatus 182. The other two unselected shim channels 86 of the retractor blades 44, 46 or 48 will engage with the two lobe features 140 and 142 of the second dilator 128 during insertion. The shim channel 86 of the selected retractor blade 44, 46 or 48 will immediately engage with the optional shim extension and/or shim component 186 along the entire length of the blade portion 50 of the retractor blade 44, 46 or 48 starting at the blade portion tip 84. In one embodiment, the medical retractor 14 can be oriented such that the retractor blade 44, 46 or 48 extending from the middle arm 18 is towards the midline of the spine 12 and the first or second outer arms 20 or 22 will engage the optional shim extension and/or shim component 186 of the threaded fixation apparatus 182. One or more of the medical retractor arms 18, 20 or 22 can be secured to the table arm if desired by the surgeon. After the medical retractor 14 is engaged with the threaded fixation apparatus 182, each of the retractor blades 44, 46 or 48 can be moved independently towards the fully open position and/or each of the retractor blades 44, 46 or 48 can be rotated (or toed) independently. The second dilator 128, the guide wire, the round body component 284 of the threaded fixation apparatus 182, and the optional shim extension of the threaded fixation apparatus 182 can be removed, leaving the shim component 226 of the threaded fixation apparatus 182 remaining engaged with the medical retractor 14. The surgeon can now perform whatever additional surgical intervention is desired.

In another embodiment of the present disclosure, the first dilator 110 can be used to target and engage the anterior third of desired disc or disc space from a lateral (LLIF) approach in a sagittal plane wherein the arc-shaped portion 122 of the first dilator 110 (the part that can engage the shim channel 86 of any of the retractor blades 44, 46 or 48) is directed anteriorly relative to the spinal anatomy. In another embodiment, the first dilator 110 can be used to target and engage the posterior third or midpoint of the desired disc space from a lateral (LLIF) approach in a sagittal plane wherein the arc-shaped portion 122 of the first dilator 110 is directed posteriorly relative to the spinal anatomy. Alternatively, in these embodiments, the patient may be placed in a lateral decubitus or prone surgical position. A guide wire 260 can be inserted through the hole that runs through the first dilator 110 or first dilator assembly to engage the desired area of the surgical site. A neuromonitoring probe can be advanced down through the outer channel 126 of the first dilator 110 as well and the first dilator 110 can be rotated about the guide wire 260 to probe for nerve proximity. The second dilator 128 can be married up to the first dilator 110 and advanced down to the desired area of the surgical site (such as to a vertebrae disposed adjacent to the targeted disc space or the disc space itself). A neuromonitoring probe could be additionally advanced down an outer channel disposed on the outer side 138 of the second dilator 128 if desired by the surgeon. The surgeon can then advance the medical retractor 14 in its closed position down over the guide wire 260, the first dilator 110 and the second dilator 128, wherein one of the shim channels 86 disposed in one of the arms (middle, first outer, or second outer 18, 20 or 22) of the medical retractor 14 engages the arc-shaped portion 122 of the first dilator 110. In an alternative embodiment, the third dilator 154 can be run down over the guide wire 260, the first dilator 110, and the second dilator 128 instead of the medical retractor 14. A neuromonitoring probe could be additionally advanced down the outer channel 180 disposed on the outer edge of the third dilator 154 if desired by the surgeon.

If the medical retractor 14 is used in this exemplary embodiment, the first dilator 110, or the first and second dilator 110 and 128, can be removed (while leaving the guide wire in place) from the medical retractor 14 so a visual inspection of the surgical site can be conducted to ensure no unexpected anatomical structures are present, such as tissue, vessels, or nerves being in the way of a disc. After visual inspection, the second dilator 128, if previously removed, can optionally be placed back down inside the retractor blades 44, 46 or 48 of the medical retractor 14 in addition to the shim fixation apparatus 222 described in its first state herein. The shim fixation apparatus 222 would be delivered over the guide wire 260 effectively re-centering the three closed retractor bladed around the guide wire. The shim fixation apparatus 222 can be transitioned to its second state where the tapered tip 256 of the shim component 226 is advanced down into the disc space to fixate the apparatus 10 to the spine 12.

If the third dilator 154 is used in this exemplary embodiment, the first dilator 110, or the first and second dilator 110 or 128, can be removed (while leaving the guide wire in place) from the third dilator 154 so a visual inspection of the surgical site can be conducted to ensure no unexpected anatomical structures are present, such as tissue, vessels, or nerves being in the way of a disc. After visual inspection, the second dilator 128, if previously removed from the third dilator 154, can optionally be placed back down inside the third dilator 154 in addition to the shim fixation apparatus 222 described in its first state herein. The shim fixation apparatus 222 would be delivered over the guide wire 260 effectively re-centering the three closed retractor bladed around the guide wire. The shim fixation apparatus 222 can be transitioned to its second state where the tapered tip 256 of the shim component is advanced down into the disc space to fixate the apparatus 10 to the spine 12. The third dilator 154 can then be removed from around the second dilator 128, the second state shim fixation apparatus 222, and the guide wire 260. The medical retractor 14 in its closed position can be advanced down into the surgical site around the guide wire 260, the second dilator 154, and the shim fixation apparatus 222 in its second state. To do this, one of the shim channels 86 in one of the retractor blades 44, 46 or 48 attached to one of the arms 18, 20 or 22 engages the shim component 226 of the shim fixation apparatus 222 such that the shim component 226 slides up and through the shim channel 86 in the desired retractor blade 44, 46 or 48. In one embodiment, the shim channel 86 in the retractor blade 44, 45 or 48 attached to the middle arm 18 is the shim channel 86 the shim component 226 of the shim fixation apparatus 222 slides up and into. The medical retractor 14 can be slid down the shim component 226 of the shim fixation apparatus 222 to a desired depth relative to the surgical site. The shim channel 86 of the of the selected retractor blade 44, 46 or 48 will immediately engage with the optional shim extension 228 and/or shim component 226 along the entire length of the blade portion 50 of the retractor blade 44, 46 or 48 starting at the blade portion tip 84. Once the medical retractor 14 is at the desired location, the shim component 226 can be locked into place relative to the shim channel 86 of the retractor blade 44, 46 or 48 that the shim component 226 is engaged with. In one embodiment, the flange 248 disposed on the slat 246 of the shim component 226 engages with one of the protrusions in the secondary channel 94 of the retractor blade 14.

One or more of the medical retractor arms 18, 20 or 22 can be secured to the table arm to fix the medical retractor 14 in the desired position. After the medical retractor 14 is engaged with the shim fixation apparatus 222, each of the retractor blades 44, 46 or 48 can be independently moved from the closed position toward an open position and or each of the retractor blades 44, 46 or 48 can be rotated (or toed) independently any desired amount by the surgeon. The two blades 44, 46 or 48 not engaging the shim fixation apparatus 222 can be secured by additional tools such as a threaded fixation pin 191 into desired parts of the spine 12, such as the superior or inferior vertebral body of the disc space being treated. The second dilator 128, the guide wire 260, the round body component 224 of the shim fixation apparatus 222, and the optional shim extension 228 of the shim fixation apparatus 222 can be removed, leaving the shim component 226 of the shim fixation apparatus 222 remaining engaged with the medical retractor 14. The surgeon can now perform whatever additional surgical intervention is desired.

Another method is contemplated using various components of the medical retractor assembly 10 described herein. In this method, the shim fixation apparatus 222 can be used by the surgeon to target a desired disc from the lateral (LLIF) approach as describe herein. The shim fixation apparatus 222 can be targeted more anteriorly along the anterior to posterior direction of the disc if desired by the surgeon whereas the shim body 234 is directed anteriorly. The guide wire 260 can be delivered to the surgical site through the shim fixation apparatus 222. The second dilator 128 can be advanced down to the surgical site around the guide wire 260 and mated up with the shim fixation apparatus 222. Similar to the method above after the third dilator 154 is removed, the medical retractor 14 can be advanced down into the surgical site around the guide wire 260, the second dilator 128 and the shim fixation apparatus 222. To do this, one of the shim channels 86 in one of the retractor blades 44, 46 or 48 attached to one of the retractor arms 18, 20 or 22 engages the optional shim extension 222 and/or shim component 226 of the shim fixation apparatus 222 such that the optional shim extensions 228 and/or shim component 226 slides up and through the shim channel 86 in the desired retractor blade 44, 46 or 48. In one embodiment, the shim channel 86 in the retractor blade 44, 46 or 48 attached to the middle arm 18 is the shim channel 86 the shim component 226 of the shim fixation apparatus 222 slides up and into. The medical retractor 14 can be slid down the shim component 226 of the shim fixation apparatus 222 to a desired depth relative to the surgical site. The remaining steps taken by the surgeon to perform a desired surgical procedure can be the same as in the previous method.

The methods described herein can also incorporate the shim tool 250 described herein to work with other components of the medical retractor assembly 10 to accomplish the desired goals of the surgeon. The shim tool 250 can potentially replace the shim fixation apparatus 222 in the methods disclosed herein. The shim tool 250 can be engaged by the medical retractor 14 via the shim channels 86 disposed on the blade portions 50 of the retractor blades 44, 46 or 48 or the second shim channel 86 embodiments disposed on the backside of the blade portion 50 of the retractor blades 44, 46 or 48. This method may also include using the shim tool 250 and one of retractor blades 44, 46 or 48 unattached to the retractor body 16 simultaneously. In this variation of the method, the shim tool 250 would be preloaded into the retractor blade shim channel 86 and the two devices would be advanced down to the targeted surgical site simultaneously. While being delivered the shim tool 250 would not be extended beyond the distal tip 84 of the retractor blade 44, 46 or 48. Once the two devices were in the desired location on the spine 12, the shim tool 250 would be advanced down further through the shim channel 86 and into the disc space where the retractor blade 44, 46 or 48 would serve to protect the tapered tip 256 of the shim tool 250 from damaging any tissue. A first dilator 110, second dilator 128, and the medical retractor 14 with two of the three middle, first outer, and second outer blades 44, 46 and 48; could then be delivered to the surgical site and attached to the existing retractor blade 44, 46 or 48 and shim tool 250.

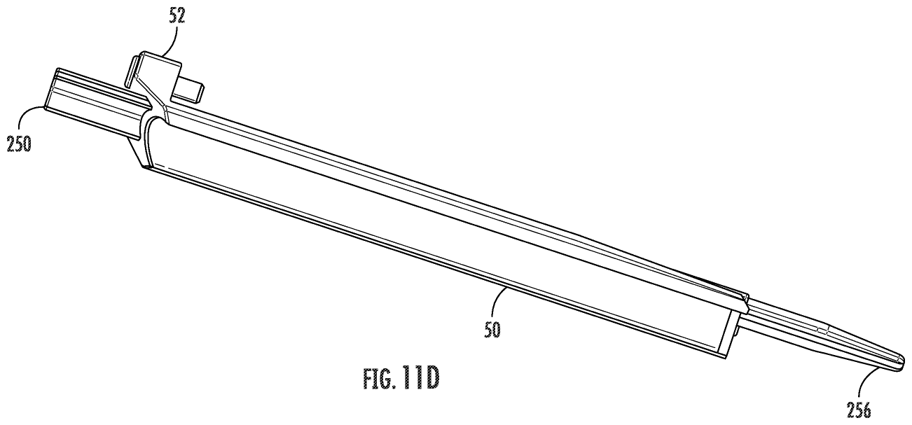

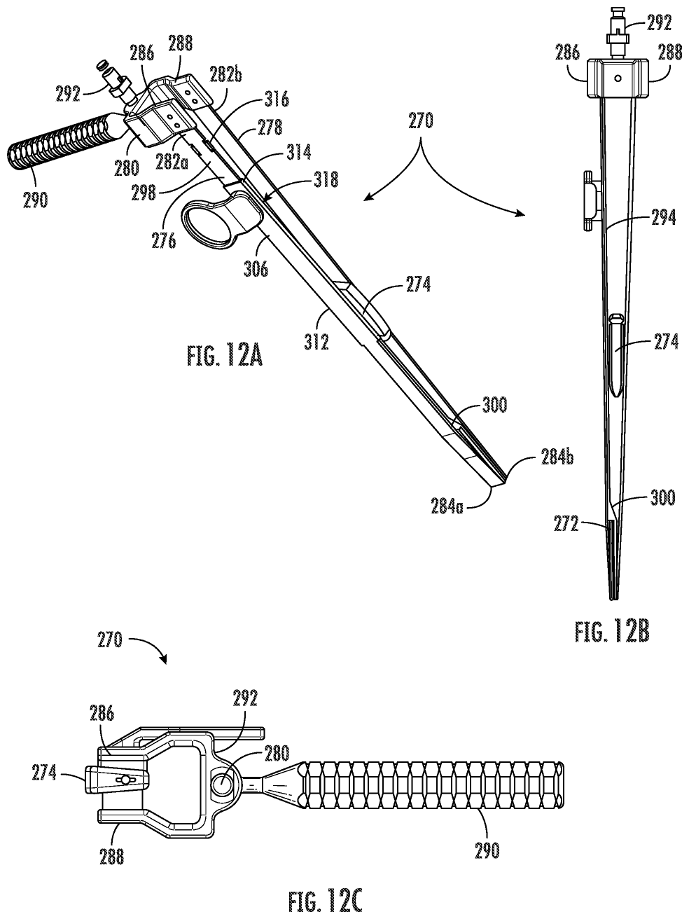

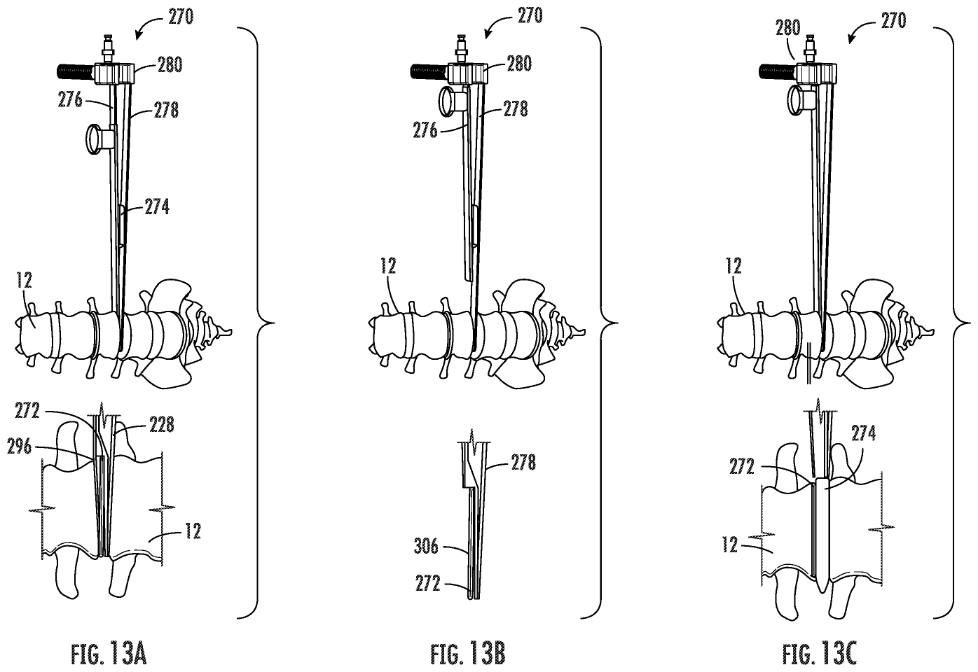

Referring now to FIGS. 12A-13C, the present disclosure is also directed to an inserter apparatus 270 that can be used with the medical retractor 14 described herein. The inserter apparatus 270 can be used to place a biological graft material 272 and an implant 274 (or spacer) at a desired location in a surgical site. The inserter apparatus 270 described herein allows a surgeon to place more than 10 cubic centimeters of graft material 272 to the disc space prior to placing and directly adjacent to the implant 274. In another embodiment, the inserter apparatus 270 allows the surgeon to place more than 15 cubic centimeters of graft material 272 to the disc space prior to placing and directly adjacent the implant 274.

The inserter apparatus 270 includes a first arm 276 and a second arm 278 that extend from a base portion 280. The first arm 276 and second arm 278 are flexible from a closed position to an open position and vice versa. The first arm 276 and the second arm 278 can each have a connection end 282a and 282b where the arms 276 and 278 are connected to the base portion 280 and a distal end 284a and 284b where the arms 276 and 278 interact with each other and the surgical site. In one embodiment, the base portion 280 of the inserter apparatus 270 can be C-shaped and have a first leg 286 and a second leg 288. The first arm 276 of the inserter apparatus 270 extends from the first leg 286 of the base portion 280 and the second arm 278 of the inserter apparatus 270 extends from the second leg 288 of the base portion 280. The C-shaped base portion 280 permits a user of the inserter apparatus 270 to be able to see between the first and second legs 286 and 288 of the base portion 280 and view the space between the first and second arms 276 and 278 of the inserter apparatus 270. The inserter apparatus 270 can also include a handle 290 that is attached to the base portion 280. The inserter apparatus 270 can also include an attachment 292 for a reverse slap hammer connection that is attached to the base portion 280.

The distal end 284a of the first arm 276 is inset from the main arm portion 294 of the first arm 276. This inset creates a shoulder 296 on the outside 298 of the first arm 276 and a transition area 300 on the inside 302 of the first arm 276. In one embodiment, the transition area 300 on the inside 302 of the first arm 276 can have an angled surface 304 to facilitate placement of the implant 274 into the disc space. The inserter apparatus 270 can also include a slide device 306 that is slidably disposed on the first arm 276. The slide device 306 can extend along the outside 298 of the first arm 276 such that a portion of the slide device 306 extends beyond the inset and creates a graft area 308 capable of holding the graft material 272. The graft area 308 is defined by the outside of the distal end 284a of the first arm 276, the inside of a distal end 310 of the slide device 306, and the shoulder 296 on the outside 298 of the first arm 276 created by the inset. The distal end 310 of the slide device 270 can extend any length beyond the shoulder 296 such that a desired size of graft material 272 can be held in the graft area 308.

The slide device 270 can include a handle for manipulating the slide device 306 relative to the first arm 276 of the inserter apparatus 270. In a first position, the distal end 310 of the slide device 306 extends beyond the shoulder 296 to maintain the graft material 272 in the graft area 308. In a second position, the slide device 306 can be moved in the direction toward the base portion 280 and secured there, which permits the graft material 272 to escape the graft area 308. In one embodiment, the distal end 310 of the slide device 306 can be bent inward relative to the main body 312 of the slide device 306 (i.e., toward the distal end 284a of the first arm 276) to better hold the graft material 272 in the graft area 308. In a further embodiment, the slide device 306 can include L-shaped flanges 314 extending from the main body 312 of the slide device 306 that engage the sides and inside 302 of the first arm 276. The slide device 306 can be secured in the second position in any manner known in the art. For example, it could be held in place by frictional engagement between the slide device 306 and the first arm 276. In another example, the sides of the first arm 276 can have a catch section 316 that engages with an engagement section 318 disposed in the L-shaped flanges 314 of the slide device 306.

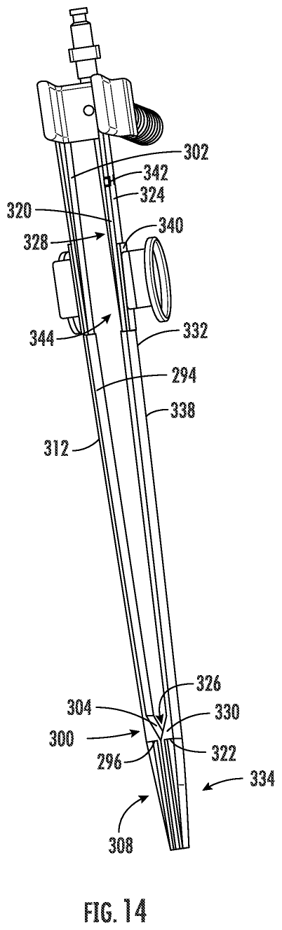

In a further embodiment of the present disclosure shown in FIG. 14, the distal end 284b of the second arm 278 is inset from the main arm 320 portion of the second arm 278. This inset creates a shoulder 322 on the outside 324 of the second arm 278 and a transition area 326 on the inside 328 of the second arm 278. In one embodiment, the transition area 326 on the inside 328 of the second arm 278 can have an angled surface 330 to facilitate placement of the implant 274 into the disc space. The inserter apparatus 270 can also include a second slide device 332 that is slidably disposed on the second arm 278. The second slide device 332 can extend along the outside 324 of the second arm 278 such that a portion of the second slide device 332 extends beyond the inset and creates a second graft area 334 capable of holding more graft material 272. The second graft area 334 is defined by the outside of the distal end 284b of the second arm 278, the inside of a distal end 336 of the second slide device 332, and the shoulder 322 on the outside of the second arm 278 created by the inset. The distal end 336 of the second slide device 332 can extend any length beyond the shoulder 322 such that a desired size of graft material 272 can be held in the second graft area 334.

The second slide device 332 can include a handle for manipulating the second slide device 332 relative to the second arm 278 of the inserter apparatus 270. In a first position, the distal end 336 of the second slide device 332 extends beyond the shoulder 322 to maintain the graft material 272 in the second graft area 334. In a second position, the second slide device 332 can be moved in the direction toward the base portion 280 and secured there, which permits the graft material 272 to escape the second graft area 334. In one embodiment, the distal end 336 of the second slide device 332 can be bent inward relative to the main body 338 of the second slide device 332 (i.e., toward the distal end 284b of the second arm 278) to better hold the graft material 272 in the second graft area 334. In a further embodiment, the second slide device 332 can include L-shaped flanges 340 extending from the main body 338 of the second slide device 332 that engage the sides and inside 328 of the second arm 278. The second slide device 332 can be secured in the second position in any manner known in the art. For example, it could be held in place by frictional engagement between the second slide device 332 and the second arm 278. In another example, the sides of the second arm 278 can have a catch section 342 that engages with an engagement section 344 disposed in the L-shaped flanges 340 of the second slide device 332.

In use, a first piece of graft material 272 can be placed in the graft area 308 for delivery to the disc space and the inserter apparatus 270 can be directed toward the surgical site. Once the first graft material 272 is in a desired position, typically between two adjacent vertebrae, the doctor will move the slide device 306 from the first position to the second position to free up the first graft material 272 to be exposed to the endplate surface of one of the adjacent vertebrae and be free to leave the graft area 308. The inserter apparatus 270 can be forced toward the spine until the shoulder created by the inset on the backside of the first arm 276 encounters the outer edge of the vertebrae the graft material 272 is intended to be placed adjacent to. The interaction of the shoulder and the vertebrae prevents the inserter apparatus 270 from going too deep into the surgical site and places the first graft material 272 in the optimum location.

In an alternative embodiment, a second piece of graft material 272 can be placed in the second graft area 334 for delivery to the disc space prior to the inserter apparatus 270 being directed toward the surgical site. Once the first graft material 272 and the second graft material 272 are in desired positions, typically between two adjacent vertebrae, the first graft material 272 can be handled as previously described herein and then the doctor can move the second slide device 332 from the first position to the second position to free up the second graft material 272 to be exposed to the surface of the vertebrae on the other side of the disc space from where the first graft material 272 was positioned. Once the second slide device 332 is shifted from the first position to the second position, the second graft material 272 is free to leave the second graft area 334. The inserter apparatus 270 can be forced toward the spine 12 until the shoulder created by the inset on the backside of the second arm 278 encounters the outer edge of the vertebrae the second graft material 272 is intended to be placed adjacent to. The interaction of the shoulder on the second arm 278 and the vertebrae helps prevent the inserter apparatus 270 from going too deep into the surgical site and places the second graft material in its optimum location.

For the following, the inserter apparatus 270 used is the embodiment described herein where the inserter apparatus 270 only has one graft area 308. An implant 274 attached to a separate implant inserter 270 can be provided to the space between the first and second arms 276 and 278 and forced, via any method known in the art, down between the first and second arms 276 and 278, passed the angled surface 307 of the transition area 300 and down between the distal ends of the first and second arms 284a and 284b. At this point, the graft material 272 is in its desired location and separated from the implant 274 by the distal end of the first arm 284a. The inserter apparatus 270 can then be withdrawn from the surgical site leaving the graft material 272 and the implant 274 adjacent to each other between two vertebrae. In some instances, the distal ends of the first and second arms 284a and 284b of the inserter apparatus 270 will need to be forcibly removed from the surgical site by attaching a reverse slap hammer to the base portion 280 of the inserter apparatus 270 while holding the implant 274 and implant inserter 270 in the desired area as the inserter apparatus 270 is withdrawn.

For the following, the inserter apparatus 270 used is the embodiment described herein where the inserter apparatus 270 has two graft areas 308 and 334. An implant 274 attached to a separate implant inserter 270 can be provided to the space between the first and second arms 276 and 278 and forced, via any method known in the art, down between the first and second arms 276 and 278, passed the angled surfaces 304 or 330 of the transition areas 300 or 326 of the first and second arms 276 and 278, and down between the distal ends of the first and second arms 284a and 284b. At this point, the first and second graft materials 272 are in their desired locations and separated from the implant 274 by the distal end of the first arm 284a and the second arm 284b, respectively. At this point, the inserter apparatus 270 can be withdrawn from the surgical site leaving the first and second graft materials 272 and the implant 274 adjacent to each other between two vertebrae. In some instances, the distal ends of the first and second arms 284a and 284b of the inserter apparatus 270 will need to be forcibly removed from the surgical site by attaching a reverse slap hammer to the base portion 280 of the inserter apparatus 270 while holding the implant 274 and implant apparatus 270 in the desired area as the inserter apparatus 270 is withdrawn.

It should be understood that it is contemplated that the inserter apparatus 270 can deposit the first graft material 272 with a first implant 274 and be moved to a second surgical area and the second graft material 272 can be deposited with a second implant 274 to the second surgical site.

From the above description, it is clear that the present disclosure is well-adapted to carry out the objectives and to attain the advantages mentioned herein as well as those inherent in the disclosure. While presently preferred embodiments have been described herein, it will be understood that numerous changes may be made which will readily suggest themselves to those skilled in the art and which are accomplished within the spirit of the disclosure and claims.

What is claimed is:

1. A medical retractor, the medical retractor comprising:
a first retractor blade with a shim channel disposed in the first retractor blade that has a cross-sectional area which remains unchanged for an entire length of the shim channel to permit medical tools to slide therethrough to engage a surgical site or to permit the medical retractor to slide down over the medical tools that are engaged with the surgical site.

2. The medical retractor of claim 1 further comprising:
a retractor body;
a first outer arm extending from the retractor body, the first retractor blade rotatably supported by the first outer arm;
a second outer arm extending from the retractor body, a second retractor blade rotatably supported by the second outer arm; and
a middle arm extending from the retractor body, a third retractor blade rotatably supported by the middle arm.

3. The medical retractor of claim 2 wherein the first retractor blade, the second retractor blade and the third retractor blade are identical.

4. The medical retractor of claim 2 further comprising:
a first threaded drive shaft disposed in the retractor body;
a first threaded shaft rotationally disposed along a first axis of the retractor body that when rotated one direction moves the middle arm along the first axis towards the retractor body and when rotated the other direction moves the middle arm along the first axis away from the retractor body;
a second threaded drive shaft in the retractor body; and
a second threaded shaft rotationally disposed along a second axis of the retractor body that when rotated one direction moves the first and second outer arms along the second axis towards each other and when rotated the other direction moves the first and second outer arms along the second axis away from each other.

5. The medical retractor of claim 4 wherein the axis of rotation of a drive tool used to rotate the first threaded shaft is in line with an axis the first threaded shaft rotates around and the axis of rotation of the drive tool used to rotate the second threaded shaft is in line with an axis the second threaded shaft rotates around.

6. The medical retractor of claim 2 wherein the first outer retractor blade and the second outer retractor blade each have an axis of rotation for each retractor blade's toeing, the axis of rotation for each retractor blade's toeing has some other relationship to an axis of translation of each retractor blade other than parallel or perpendicular.

7. The medical retractor of claim 2 wherein the second retractor blade and the third retractor blade each have a shim channel disposed therein that has a cross-sectional area which remains unchanged for an entire length of the shim channel.

8. The medical retractor of claim 2 wherein the first retractor blade, the second retractor blade and the third retractor blade each have a back shim channel disposed on back sides of each retractor blade.

9. A medical retractor assembly, the medical retractor assembly comprising:
a medical retractor having a first retractor blade with a shim channel disposed in the first retractor blade; and
a first dilator having a cylindrical component and a shim component wherein the shim component can slidably engage with the shim channel of the medical retractor, the first dilator having a cross-section for most of its length that has a round portion and an arc-shaped portion attached to the round portion.

10. The medical retractor assembly of claim 9 wherein the medical retractor further comprises:
a retractor body;
a first outer arm extending from the retractor body, the first retractor blade rotatably supported by the first outer arm;
a second outer arm extending from the retractor body, a second retractor blade rotatably supported by the second outer arm; and
a middle arm extending from the retractor body, a third retractor blade rotatably supported by the middle arm.

11. The medical retractor assembly of claim 10 wherein the second retractor blade and the third retractor blade each have a shim channel disposed therein that has a cross-sectional area which remains unchanged for an entire length of the shim channel.

12. The medical retractor assembly of claim 11 wherein the first dilator includes an inner member that is slidably disposed within an outer member, the inner member having a central hole running axially therethrough to permit engagement with a guide wire and the outer member having an outer profile that creates the round portion and the arc-shaped portion.

13. The medical retractor assembly of claim 12 further comprising a second dilator that is generally crescent-moon shaped wherein an inner portion thereof can receive the round portion of the first dilator and ends of the second dilator can abut the arc-shaped portion of the first dilator.

14. The medical retractor assembly of claim 13 wherein the second dilator includes a first lobe and a second lobe that can slidably engage the shim channels of the second retractor blade and the third retractor blade.

15. The medical retractor assembly of claim 14 further comprising a third dilator that includes at least one shim channel disposed axially therein for receiving the arc-shaped portion of the first dilator, the first lobe of the second dilator or the second lobe of the second dilator.

16. The medical retractor assembly of claim 15 wherein an inner, cross-sectional profile of the third dilator matches an inner, cross-sectional profile of the first, second and third retractor blades when the medical retractor is in a fully closed position.

17. The medical retractor assembly of claim 14 wherein a series of concentric circles are generated by the inner member of the first dilator, the arc-shaped portion of the first dilator, the first and second lobes of the second dilator and base portions of the shim channels disposed in the first, second and third retractor blades.

18. The medical retractor assembly of claim 10 further comprising:
a cross-bar attachable to the first and second outer arms to securely hold other medical tools that can be used with the medical retractor assembly;
a slot disposed in the cross-bar to permit movement of the first and second outer arms while the cross-bar is attached to the medical retractor; and
a blade retainer attached to a back side of the cross-bar and a retention device extending through an opening in the cross-bar wherein the retention device can engage medical tools and secure them inside the blade retainer.

19. The medical retractor assembly of claim 9 further comprising a threaded fixation apparatus, the threaded fixation apparatus comprising:
an outer assembly that has a round body component and a shim component for accessing the shim channel in the first retractor blade, the outer assembly having a cross-sectional shape that is similar to the first dilator;
an inner body that can be disposed in the outer body;
a fixation pin support member attachable to the inner body that holds a threaded fixation pin; and
a driving apparatus that can extend within the inner body, the driving apparatus has a drive end to engage the threaded fixation pin to rotatingly advance the threaded fixation pin into a surgical site.

20. The medical retractor assembly of claim 11 further comprising a shim fixation apparatus that has a round body component and a shim component, the shim component slidably engageable with and through the entire length of the shim channel disposed in the first retractor blade, the shim fixation apparatus has a cross-sectional shape that is similar to a cross-sectional shape of the first dilator.
